(19) United States
(12) Patent Application Publication
(10) Pub. No.: US 2014/0074126 A1
(43) Pub. Date: Mar. 13, 2014

(54) REMOVING NEEDLES FROM A SUTURING DEVICE

(71) Applicants: Laveille Kao Voss, Belmont, CA (US); Wouter E. Roorda, Palo Alto, CA (US); Stephanie Henze, San Mateo, CA (US); Kristopher M. Konawalik, San Francisco, CA (US); Leah M. Davis, San Francisco, CA (US); Aaron M. Fortson, Fremont, CA (US)

(72) Inventors: Laveille Kao Voss, Belmont, CA (US); Wouter E. Roorda, Palo Alto, CA (US); Stephanie Henze, San Mateo, CA (US); Kristopher M. Konawalik, San Francisco, CA (US); Leah M. Davis, San Francisco, CA (US); Aaron M. Fortson, Fremont, CA (US)

(73) Assignee: ABBOTT CARDIOVASCULAR SYSTEMS, INC., Santa Clara, CA (US)

(21) Appl. No.: 13/610,595

(22) Filed: Sep. 11, 2012

(51) Int. Cl. A61B 17/06; A61B 17/04

(52) U.S. Cl. CPC A61B 17/06161; A61B 17/0482
USPC 606/148; 606/223

(57) ABSTRACT

Suturing systems and needle removal devices for more efficiently removing needles from suturing systems are provide. In some embodiments, a suturing system guide body includes one or more needle lumens that transfer corresponding needles circumferentially around the guide body. Thus, when the needles exit the needle lumens at the proximal end of the guide body, the needles are in closer proximity to one another for removal. Other embodiments include various needle capture devices tools used for removing needles from a suturing system. Needle capture devices and tools can include needle receptacles positioned and configured to generally correspond to the needle lumens of the suturing system. The needle receptacles may be further configured to selectively receive and grasp onto one or more needles extending proximally from the needle lumens of the suturing system.

### CROSS-REFERENCE TO RELATED APPLICATIONS

Not Applicable

### BACKGROUND OF THE INVENTION

1. The Field of the Invention

The present invention relates generally to devices, systems, and methods for removing needles from devices or systems used to close openings in body lumens. More particularly, the present invention relates to devices, methods, and systems for removing needles from systems or devices used for closure of arterial and venous puncture sites accessed through a tissue tract.

2. The Relevant Technology

A number of diagnostic and interventional vascular procedures are now performed translumenally. A catheter is introduced to the vascular system at a convenient access location and guided through the vascular system to a target location using established techniques. Such procedures require vascular access, which is usually established using the well-known Seldinger technique. Vascular access is generally provided through an introducer sheath, which is positioned to extend from outside the patient's body into the vascular lumen. When vascular access is no longer required, the introducer sheath is removed and bleeding at the puncture site stopped.

One common approach for achieving hemostasis (the cessation of bleeding) is to apply external force near and upstream from the puncture site, typically by manual compression. However, the use of manual compression suffers from a number of disadvantages. For example, the manual compression procedure is time consuming, frequently requiring one-half hour or more of compression before hemostasis is achieved. Additionally, such compression techniques rely on clot formation, which can be delayed until anticoagulants used in vascular therapy procedures (such as for heart attacks, stent deployment, non-optical PTCA results, and the like) wear off. The anticoagulants may take two to four hours to wear off, thereby increasing the time required before completion of the manual compression procedure.

Further, the manual compression procedure is uncomfortable for the patient and frequently requires analgesics to be tolerable. Moreover, the application of excessive pressure can at times totally occlude the underlying blood vessel, resulting in ischemia and/or thrombosis. Following manual compression, the patient typically remains recumbent from four to as much as twelve hours or more under close observation to assure continued hemostasis. During this time, renewed bleeding may occur, resulting in blood loss through the tract, hematoma and/or pseudo-aneurysm formation, as well as arteriovenous fistula formation. These complications may require blood transfusions and/or surgical intervention.

The incidence of complications from the manual compression procedure increases when the size of the introducer sheath grows larger, and/or when the patient is anticoagulated. The compression technique for arterial closure can be risky, and is expensive and onerous to the patient. Although trained individuals can reduce the risk of complications, dedicating such personnel to this task is both expensive and inefficient. Nonetheless, as the number and efficacy of translumenally performed diagnostic and interventional vascular procedures increases, the number of patients requiring effective hemostasis for a vascular puncture continues to increase.

To overcome the problems associated with manual compression, the use of bioabsorbable sealing bodies is another example approach that has been proposed to achieve hemostasis. Generally, the use of bioabsorable sealing bodies relies on the placement of a thrombogenic and bioabsorbable material, such as collagen, at the superficial arterial wall over the puncture site. While potentially effective, the use of bioabsorbable material suffers from a number of drawbacks. For example, bioabsorbable sealing bodies may lack a solid mechanical attachment of the sealing body to the tissue. Due to the lack of a solid mechanical attachment, the sealing body can wander within the tissue tract or move out of the puncture site, thus causing late bleeds. Conversely, if the sealing body wanders and intrudes too far into the arterial lumen, due to the lack of a solid mechanical attachment, intravascular clots and/or collagen pieces with thrombus attached can form and embolize downstream, causing vascular occlusion.

In addition to not having a solid mechanical attachment to the tissue, the sealing bodies may rely upon expandable materials to achieve hemostasis. Again, the expandable materials lack the security of a hard mechanical closure, thus potentially causing late bleeds and prolonging hemostasis.

A further approach to achieving hemostasis is to use a suture to close a puncture site. Although difficult to suture manually, suture applying devices can be used to appropriately place a suture for closing a puncture site. One example suture applying device has a shaft carrying a pair of needles near its distal end. The needles are joined together by a length of suture. The shaft is used to introduce the needles into a lumen of a body structure and the needles pushed back through the lumen wall on either side of a puncture site. After the needles have passed back through the tissue, they are captured on the shaft and drawn proximally away from the body structure. Drawing the needles outward leaves a loop of suture behind to close the puncture site. The loop of suture can then be tied in a knot to complete the closure. Suture applying devices address many disadvantages associated with to use of external force (e.g., digital compression and with the use of bioabsorbable sealable bodies to achieve hemostasis.

However, the use of suture applying devices also has a number of inefficiencies. Typically, to access a suture in manner that it can be tied off, the needle must be fully removed from the shaft and other components subsequently moved out of the way. However, after needle deployment, suture applying devices are often configured to draw needles proximally only to a point where they are partially exposed at the proximal end of the shaft. To remove needles from the shaft completely, an operator has to use manual force to individually grab the proximal end of the each needle (e.g., with a hemostat) and draw it further proximally while also securely holding the shaft. The amount of force required to further draw the needle proximally can sometimes be quite large (and potentially unacceptable).

Some suture applying devices have a separate internal needle holder that can be used to grab a partially exposed needle. The needle holder assists an operator in drawing the needle proximally until the distal end of the needle exits the proximal end of the shaft. However, needle holders often do not sufficiently grip a needle such that it can be efficiently drawn proximally. Additionally, the leverage obtained from using a needle holder is often insufficient to remove a needle from challenging (e.g., calcified or scarred) tissue anatomy.

For at least these reasons, it would be desirable to provide devices and methods for more efficiently removing needles from a suture applying device. It would be particularly desirable to provide devices and methods for efficiently removing needles form a suture applying device used to suture a puncture site associated with a percutaneous vascular procedure.

### BRIEF SUMMARY OF THE INVENTION

The present invention relates to methods and devices for removing needles from suturing systems and devices used to close openings in body lumens. In one embodiment, a suturing device has a guide body with a slidably mounted shaft. A plurality of needles is carried near the distal end of the shaft. One or more lengths of suture secured to and extending between the plurality of needles.

A plurality of need lumens, each having a distal end opening and corresponding proximal end opening on the guide body, is included in the guide body. Each needle lumen is configured to receive a corresponding one of the plurality of needles at a distal end opening and transfer the needle to the corresponding proximal end opening as the plurality of needles are drawn proximally by the shaft. Each of the distal end openings and proximal end openings are located outwardly from the center of the guide body. The proximal end openings are clustered together in closer proximity to one another relative to the distal opening. The pat of at least one needle lumen is partially circumferentially around the guide body to move at least one needle into a proximal end opening when the plurality of needles are drawn proximally by the shaft.

Other embodiments include need removal devices. Needle removal devices can include a body member configured to be positioned on one or more proximally exposed needles of a suturing system. The one or more needles are exposed from corresponding needle lumens on a proximal portion of a suturing system. One or more needle receptacles are at least partially defined by the body member. The one or more needle receptacles are positioned and configured to generally correspond to the needle lumens of the suturing device. The needle receptacles are further configured to selectively receive and grasp onto the proximally exposed one or more needles.

Accordingly, needle removal devices can be used to remove needles from a suturing system. A plurality of needles is drawn proximally through the suturing system until at least tips of the plurality of needles exit from the proximal end of the suturing system. A needle removal device is positioned substantial adjacent to a proximal end of the suturing system. At least the tips of one or more of the needles are received within needle receptacles formed within the needle removal device to secure the tips of the one or more needles within the needle receptacles. The need removal device is moved proximally relative to the suturing system to remove the one or more needles from the suturing system.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1A illustrates a side perspective view of a suturing system.

FIG. 1B illustrates a more detailed side perspective view of a more proximal end of the suturing system of FIG. 1A.

FIG. 1C illustrates a more detailed view of a portion of the suturing system of FIG. 1A taken along line 1C.

FIG. 1D illustrates a cross-sectional view of the suturing system of FIG. 1A taken along line 1D-1D.

FIG. 1E illustrates a cross-sectional view of the suturing system of FIG. 1A taken along line 1E-1E.

FIG. 1F illustrates a cross-sectional view of the suturing system of FIG. 1A taken along line 1F-1F.

FIGS. 2A-2C illustrate exemplary steps for pulling needles proximally into a position that the needles can be removed from the suturing system of FIG. 1A.

FIGS. 3A-3B illustrate a partial top view of a suturing system.

FIGS. 3C-3D illustrate a side cut away view of the suturing system from FIGS. 3A-3B

FIG. 4A illustrates a perspective view of a suturing system;

FIG. 4B illustrates a side view of the suturing system shown in FIG. 4A;

FIG. 4C illustrates a perspective view of needle removal device for removing needles from a suturing system;

FIG. 4D illustrates a cross-sectional view of the needle removal device shown in FIG. 4D taken along line 4D-4D;

FIG. 4E illustrates a cross-sectional view of the needle removal device shown in FIG. 4E taken along line 4E-4E;

FIGS. 4F-4G illustrates a side view of the suturing system shown in FIG. 4A with needles captured in the needle removal device shows in FIG. 4C;

FIG. 5A illustrates a side partial cross-section view a suturing system;

FIG. 5B illustrates an isometric view of a needle removal device for use with the suturing system of FIG. 5A;

FIG. 5C illustrates the needle removal device of FIG. 5B attached to the suturing system of FIG. 5A;

FIGS. 5D-5F illustrates a perspective view of the suturing system shown in FIG. 5A with needles captured in the needle removal device shown in FIG. 5B;

FIGS. 5G-5I illustrates a perspective view of another suturing system with needles captured in needle removal devices shown in FIG. 5B;

FIG. 6A illustrates a needle removal device;

FIG. 7A illustrates a needle remove device;

FIG. 7B illustrates a needle remove device;

FIGS. 7C-7E illustrate mechanisms for capturing needles.

### DETAILED DESCRIPTION

As used herein, the term “distal” is generally defined as in the direction of the patient or away from a user of a device. In the context of a medical device intervention with or through a vessel wall, “distal” herein refers to the interior or the lumen side of the vessel wall. Conversely, “proximal” generally means away from the patient or toward the user. In the context of a medical device intervention with or through a vessel wall, “proximal” herein refers to the exterior or outer side of the vessel wall.

The term “hemostasis” is herein used to mean the arrest of bleeding or substantially blocking flow of blood outwardly from a vessel lumen while the vessel lumen is pressurized or sustaining physiological blood flow. This amount of blockage or occlusion to flow is further defined such that the blood loss which is experienced is less than an amount which would affect procedural methods or outcomes according to a physician user of a device of ordinary skill in the art. In other words, “hemostasis” is not intended to mean only “total hemostasis” such that there is a total lack of blood loss. Rather, the term is used to also mean “procedural hemostasis” as a relative term in its use among physicians of ordinary skill.

The term “suturing” is herein intended to include the process of joining two surfaces or edges together with a suture such as a thread of material (either polymeric or natural), gut, wire, or the like or so as to close an aperture, opening, or wound, or join tissues.

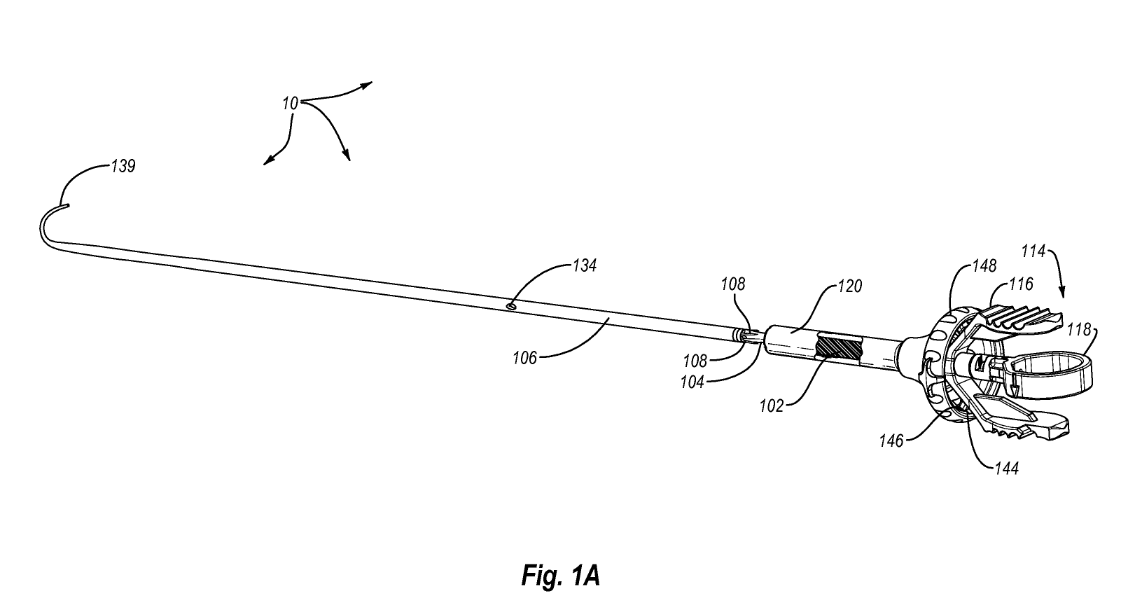

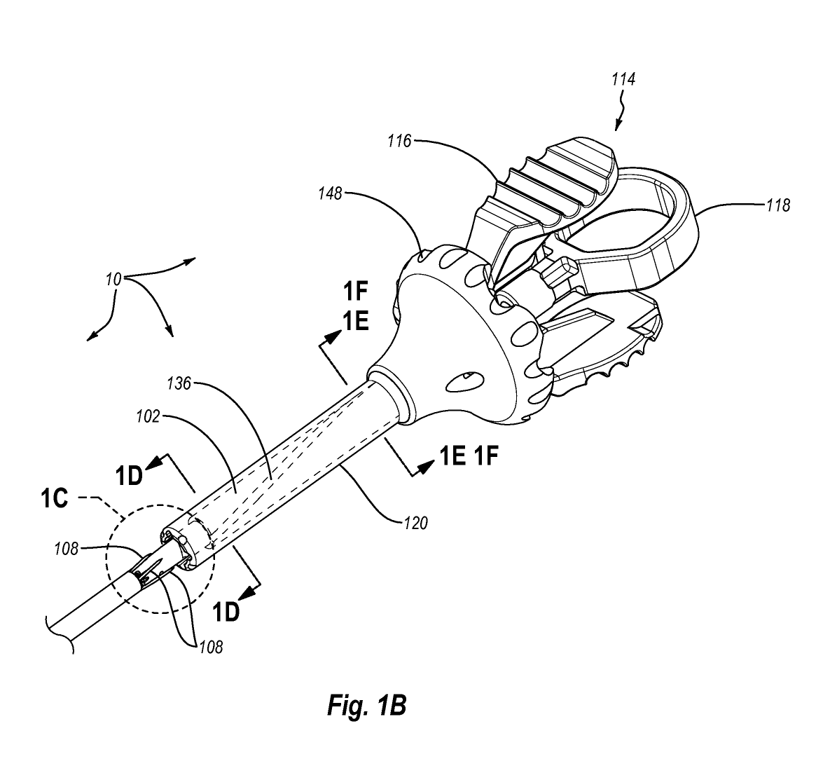

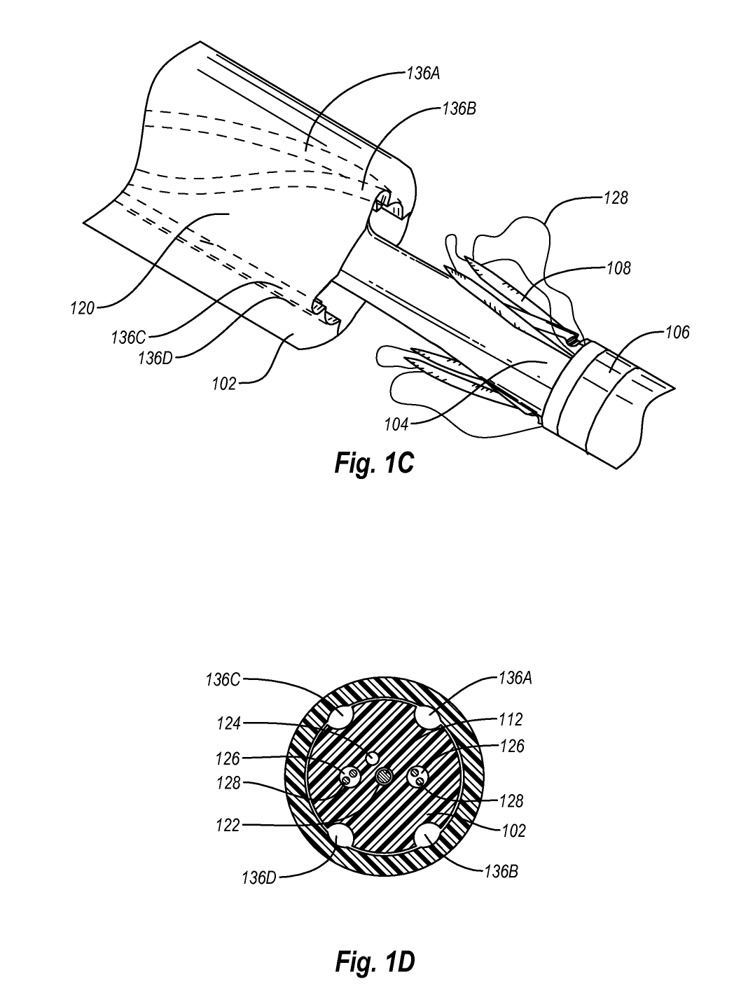

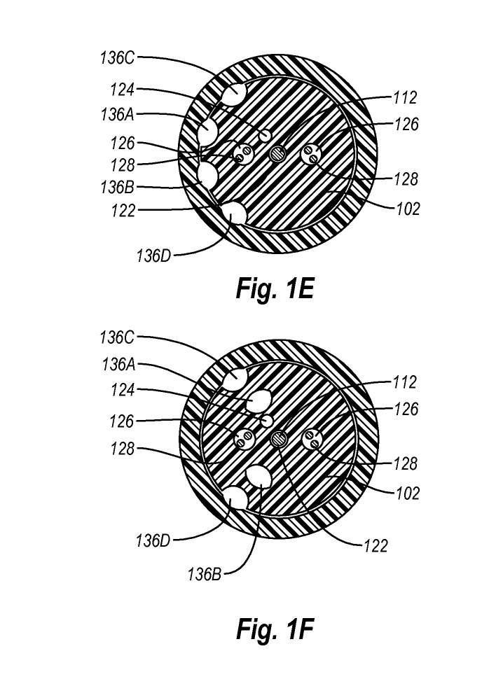

FIG. 1A illustrates a side perspective view of suturing system 10. Referring to FIG. 1A, suturing system 10 may be provided to close openings in body tissues. Suturing system 10 can include guide body 102, needle guide 104 secured to a distal end of guide body 102, and flexible tube 106 secured to a distal end of needle guide 104. Handle assembly 114 may be attached to a proximal end of guide body 102. Sheath 120 may also be rotatably received over guide body 102. Sheath 120 may be sized to be introducible through the percutaneous tissue tract. Sheath 120 may be inflexible or flexible and formed at least partially from metal, a hard plastic or polymer material, or other suitable materials.

Handle assembly 114 may include interlock wings 116 and handle 118. Interlock wings 116 can each include a detent 144 for engaging a pair of grooves 146 in hub 148 of sheath 120. Interlock wings 116 may be constructed of a resilient material (e.g., polycarbonate) so that interlock wings 116 may be pressed together to remove the detents 144 from grooves 146. Upon removal of the detents 144 from grooves 146, sheath 120 may be rotated relative to guide body 102 by maintaining a grip on interlock wings 116 with one hand and rotating hub 148 with the other hand. In other embodiments, interlock wings 116 and hub 148 may allow a physician or other user to hold and manipulate suturing system 10. For example, a physician can hold on to hub 148 when inserting and withdrawing suturing system 10 from a puncture site.

Flexible tube 106 may be formed from a flexible plastic, polymer, metal, combinations thereof, or any other suitable material. Flexible tube 106 may be generally circular in cross-sectional geometry and may include a guide wire lumen (not shown) and the central lumen (not shown) configured to house the support holster (not shown) and a plurality of needles 108 (hereinafter referred to as needles 108). Flexible tube 106 may further include guide wire exit port 134 configured to allow a guide wire that is advanced proximally through a guide wire lumen (not shown) to exit from a side of flexible tube 106. Optionally, flexible tube 106 may include distal J-tip 139 for atraumatic tracking through vessels or other body lumens. In other embodiments, distal J-tip 139 may be omitted.

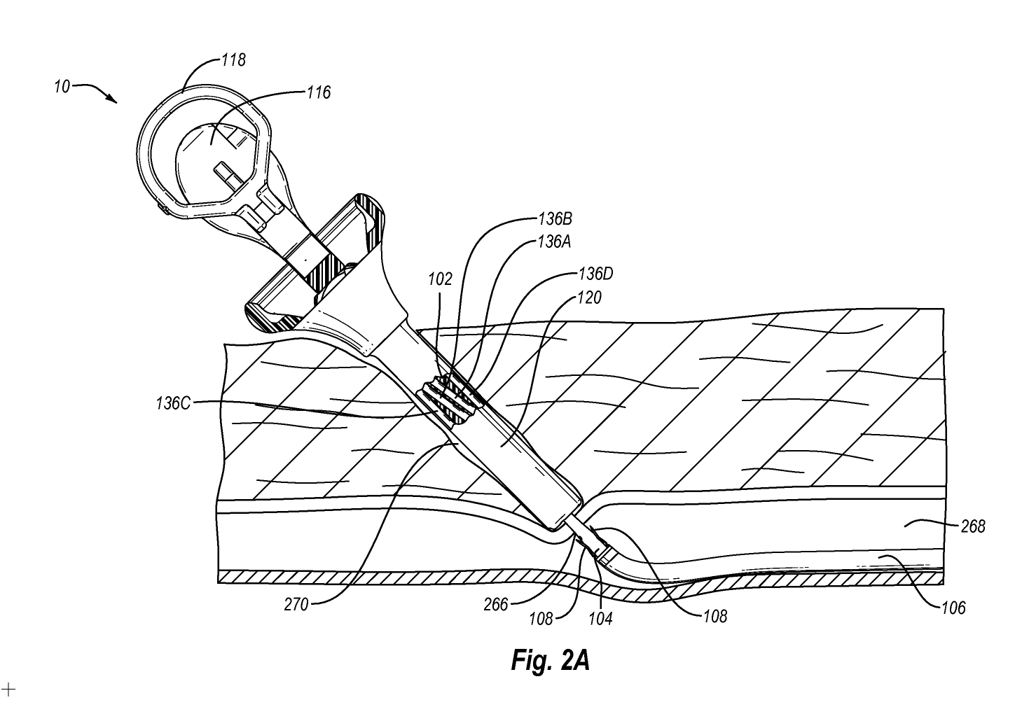

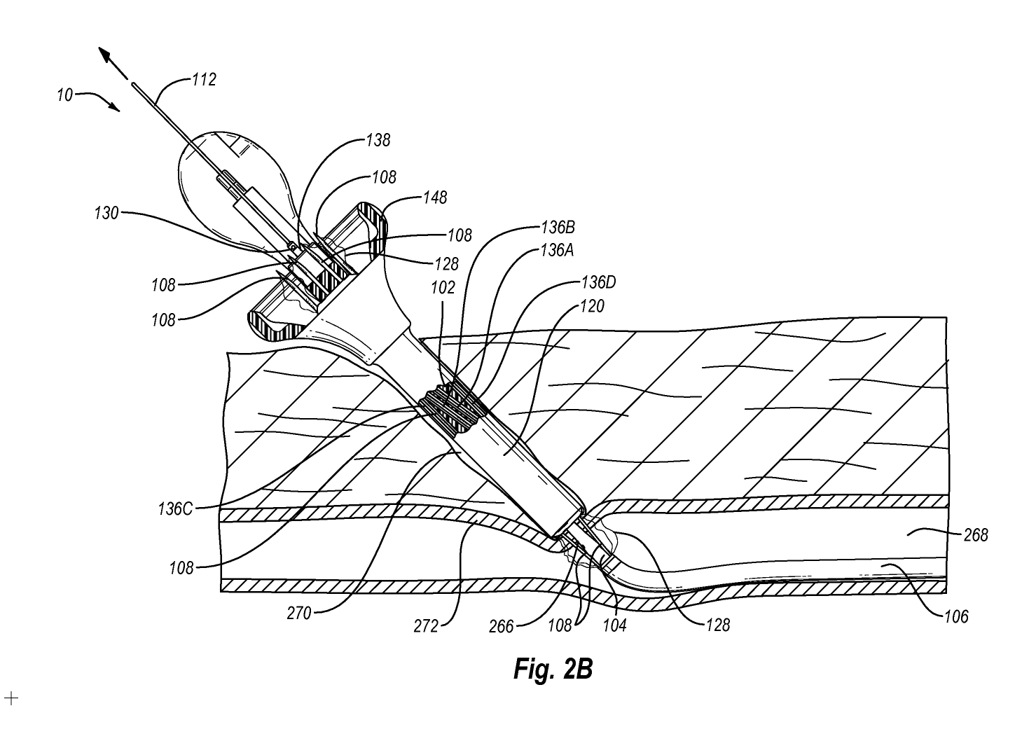

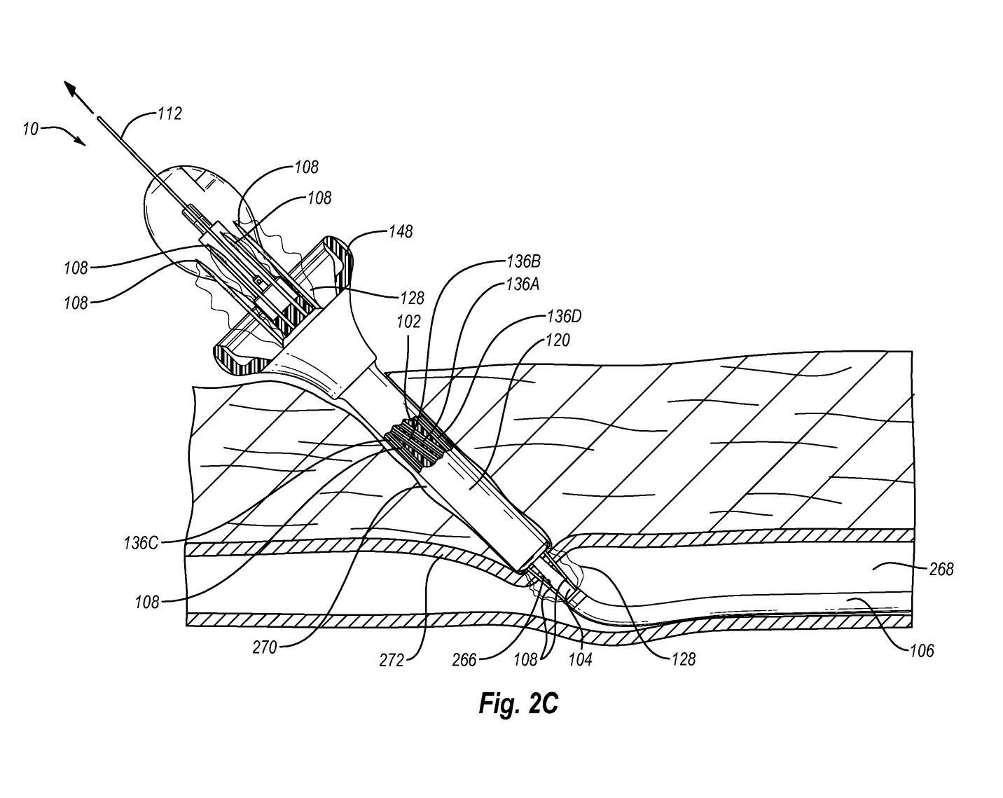

FIG. 1B illustrates a more detailed side perspective view of a more proximal end of suturing system 10. Turning to FIG. 1B, needles 108 may be mounted with their distal ends in a support holster (not shown) within flexible tube 106. In one embodiment, handle 118 is connected to a proximal end of moveable needle deployment shaft 112 (depicted in FIG. 2B) and needles 108 are connected to a distal end of moveable needle deployment shaft 112. As such, handle 118 can be pulled proximally to actuate needle deployment shaft 112 and thus also pull needles 108 proximally.

Still referring to FIG. 1B, guide body 102 may further include a plurality of needle lumens 136. Each of the plurality of needle lumens 136 can have a distal end opening on guide body 102 and a corresponding proximal end opening on guide body 102. Needles 108 may enter the distal ends of needle lumens 136 after needles 108 exit needle guide 104. As such, each of the plurality of needle lumens 136 is configured to receive a corresponding one of needles 108 at a distal end opening. The plurality of needles 108 are transferred to the corresponding proximal end opening as needles 108 are drawn proximally, for example, by moveable needle deployment shaft 112

FIG. 1C illustrates a more detailed view of a portion of the suturing system 10 taken along line 1C. Referring to FIG. 1C, the plurality of needle lumens 136 are individual referenced as needle lumens 136A, 136B, 136C, and 136D. When needle deployment shaft 112 is moved proximally relative to guide body 102, needles 108 may be drawn proximally through flexible tube 106, out needle guide 104 and toward guide body 102. Needles 108 may carry suture lengths 128. Suture lengths 128 can be used to close a puncture site. As needles 108 extend from needle guide 104, needles 108 may pass through tissue positioned between needle guide 104 and guide body 102. Guide body 102 may then capture the needles 108 (at the distal end of needle lumens 136A, 136B, 136C, and 136D) and route needles 108 proximally toward a user of suturing system 10.

At the distal end of sheath 120, each of needle lumens 136A, 136B, 136C, and 136D may be axially aligned and circumferentially spaced about the periphery of guide body 102. For example, each of the distal end openings needle lumens 136A, 136B, 136C, and 136D can be located outwardly from the center of guide body 102 and spaced essentially equidistant from one another on the distal end of guide body 102. Each of the proximal end openings of needle lumens 136A, 136B, 136C, and 136D can also be located outwardly from the center of guide body 102. Within guide body 102, needle lumens 136A and 136B can be configured to transfer corresponding needles 108 circumferentially when corresponding needles 108 are drawn proximally.

FIG. 1D illustrates a cross-sectional view of the suturing system of FIG. 1A taken along line 1D-1D. Referring to FIG. 1D, an arrangement of the distal ends for lumens 136A, 136B, 136C, and 136D is depicted. Guide body 102 may also define one or more other axial lumens or channels therein. For example, central axial lumen 122 may be provided for slidably receiving needle deployment shaft 112. Guide body 102 may also include one or more blood detection lumens 124 and one or more suture lumens 126 (for sutures 128) that pass there through.

One or more blood detection lumens 124 may be configured for receiving blood from the vessel to assist in positioning suturing system 10. One or more suture lumens 126 may be configured to receive suture lengths 128 attached to needles 108. In other embodiments, blood detection lumen 124 may be omitted. As depicted in FIG. 1D, needle lumens 136A, 136B, 136C, and 136D are positioned outwardly from central axial lumen 122 and are spaced roughly equidistant from one another on guide body 102.

Central axial lumen 122 may extend from flexible tube 106, through needle guide 104, through guide body 102 and into stem 138 (see FIG. 2B) of handle assembly 114. Needle deployment shaft 112 may run the length of central lumen 122. Accordingly, handle 118 may be proximally moved to move needle deployment shaft 112 through central lumen 122 which in turn moves needles 108. One or more suture lumens 126 may run generally parallel or non-parallel to central lumen 122. Suture lengths 128 may pass through one or more suture lumens 126.

In one embodiment, suture lengths 128 may be configured in the form of the loop with the free ends being attached to needles 108 and with the looped end passing outside suturing system 10 through the tube (not shown). Such a configuration facilitates management of suture lengths 128 during insertion of suturing system 10 to a puncture site and during movement of needles 108 to suture the vessel wall. As needles 108 are proximally advanced through guide body 102, suture lengths 128 are drawn distally through suture lumen 126 where they are completely removed from suture lumen 126 upon full deployment of needles 108 wherein the tips of needles 108 exit hub 148

FIG. 1E illustrates a cross-sectional view of the suturing system of FIG. 1A taken along line 1E-1E. Referring now to FIG. 1E, an arrangement of proximal ends for lumens 136A, 136B, 136C, and 136D is depicted. As described, needle lumen 136A and need lumen 136B can be configured to transfer a needle 108 radially around guide body 102. Thus, when needles 108 come out of the proximal end of guide body 102, the needles 108 are in closer proximity to one another. As such, needles 108 are easier to completely remove from guide body 102.

FIG. 1F illustrates a cross-sectional view of the suturing system of FIG. 1A taken along line 1F-1F. Referring now to FIG. 1F, another arrangement of the proximal ends for lumens 136A, 136B, 136C, and 136D is depicted. Similar to the arrangement in FIG. 1E, when needles 108 come out of the proximal end of guide body 102, the needles 108 are in closer proximity to one another and are easier to completely remove from guide body 102.

FIGS. 2A-2C illustrate exemplary steps for pulling needles proximally into a position that the needles can be removed from suturing system 10. Referring to FIGS. 2A-2C, guide body 102 of suturing system 10 may be introduced within a percutaneous tissue tract leading to a puncture site with flexible tube 106 positioned within a vessel. Although suturing system 10 is depicted, it is to be appreciated that the described method may utilize any other suturing system or system disclosed herein. Moreover, for ease of reference, one of interlock wings 116 has been removed from suturing system 10.

Referring now more specially to FIG. 2A, suturing system 10 can be advanced through an access tract 270 to position needles 108 encased by flexible tube 106 within vessel 268 past puncture site 266. In other embodiments, suturing system 10 may be introduced over a guide wire (not shown) passing through vessel 268. For example, an introducer sheath (not shown) may be placed over a guide wire passing percutaneously beneath the patient's skin. The introducer sheath may then be withdrawn from puncture site 266 by sliding the introducer sheath over the guide wire. As depicted, needle lumens 136A, 136B, 136C, and 136D are contained in guide body 102.

Suturing system 10 may then be introduced over the guide wire by passing the guide wire proximally through flexible tube 106 until the guide wire exits at exit port 134 (e.g., as depicted in FIG. 1A). Flexible tube 106 may then be further advanced over the guide wire until needle guide 104 is about to enter access tract 270. At this point, the guide wire is pulled from flexible tube 106 and is withdrawn from puncture site 266. With the guide wire removed, suturing system 10 may be further advanced into vessel 268 to pass needle guide 104 through access tract 270 into vessel 268.

Turning to FIG. 2B, additional components of suturing system 10 are depicted. Stem 138 may be formed between interlock wings 116 for receiving handle 118. Stem 138 may include key 130 that is received into a slot (not shown) in handle 118. Such a configuration may allow handle 118 to be slid into stem 138 with key 130 being received into the slot. Handle 118 may be rotated in a clockwise direction to secure handle 118 to the stem and prevent axial translation of needle deployment shaft 112. To move needle deployment shaft 112 and deploy needles 108, handle 118 may be rotated in a counter-clockwise direction so that key 130 may be pulled from the slot. Handle 118 may then be proximally moved to deploy needles 108.

Handle assembly 114 may be securely attached to guide body 102 so that sheath 120 may be rotated relative to guide body 102 when holding handle assembly 114. Handle assembly 114 may be securely fastened to guide body 102 by gluing, molding, and the like. In other embodiments, handle assembly 114 may be formed as an integral part of guide body 102. Handle assembly 114 may also include a plurality of tubes (not shown) aligned with blood detection lumen 124 and one or more suture lumens 126. At least a portion of the suture lengths may pass through one or more of the tubes.

Still referring to FIG. 2B, to deploy needles 108, handle 118 may be drawn proximally relative to guide body 102 to proximally move needle deployment shaft 112. In other embodiments, handle 118 may be rotated counter-clockwise to disengage key 130 from the slot in stem 138 prior to drawing handle 118 proximally. As shown, needles 108 will exit from needle guide 104, pass through vessel wall 272, and be directed toward needle lumens 136 of guide body 102.

As needles 108 are drawn through vessel wall 272, suture lengths 128 will be fed distally through one or more suture lumens 126 (e.g., as depicted in FIG. 1C). Needles 108 will then be advanced into needle lumens 136, with suture lengths 128 being continually fed through one or more suture lumens 126. Handle 118 may continue to be drawn proximally (i.e., outward from the patient) in order to continue to pull needles 108 through guide body 102. Proximal movement of needles 108, in turn, continues to draw needles 108 proximally (and for needles 108 in needle lumens 136A and 136B also circumferentially) through needle lumens 136. Eventually, needles 108, with suture lengths 128 still attached thereto, exit hub 148.

Thus, lumens 136A, 136B, 136C, 136D can be arranged on the distal end of guide body 102 for effective hole closure. Due at least in part to the circumferential path of needle lumens 136A and 136B, needles 108 exit hub 148 in closer proximity to one another than when needles 108 were captured into the distal end of guide body 102. Since needles 108 are in closer proximity to one another, needles 108 can be more easily removed from suturing system 10. Advantageously, suturing system 10 provides effective hole closure together with more efficient needle removal.

Turning to FIG. 2C, FIG. 2C depicts a proximal extent to which needles 108 can be pulled by needle deployment shaft 112. Other techniques (e.g., hemostat, needle removal devices, etc.) can be used to fully remove needles 108 from suturing system 10 and make suture lengths 128 available to a user.

Once needles 108 are removed from guide body 102, slack may be removed from suture lengths 128 by pulling them to evenly matched lengths and tensioning until resistance is felt. Suture lengths 128 may then be cut substantially close to needles 108 and needles 108 may be disposed of. Suturing system 10 may then be removed from access tract 270 to allow closure of puncture site 266. Such a configuration of suturing system 10 may allow a user to safely and securely close a puncture site.

In other embodiments, suturing system 10 may be readily adapted for use with punctures made to a variety of hollow body organs and lumens. It may, however, be necessary to modify the dimensions and other particular aspects of suturing system 10 to accommodate the different usage environments. For example, the distance between needle guide 104 and guide body 102 may be configured to allow transapical insertion of suturing system 10 into a heart ventricle as described in U.S. patent application, entitled “Apparatus and Method for Suturing Body Lumens,” attorney docket number 16497.229, the disclosure of which is incorporated herein in its entirety.

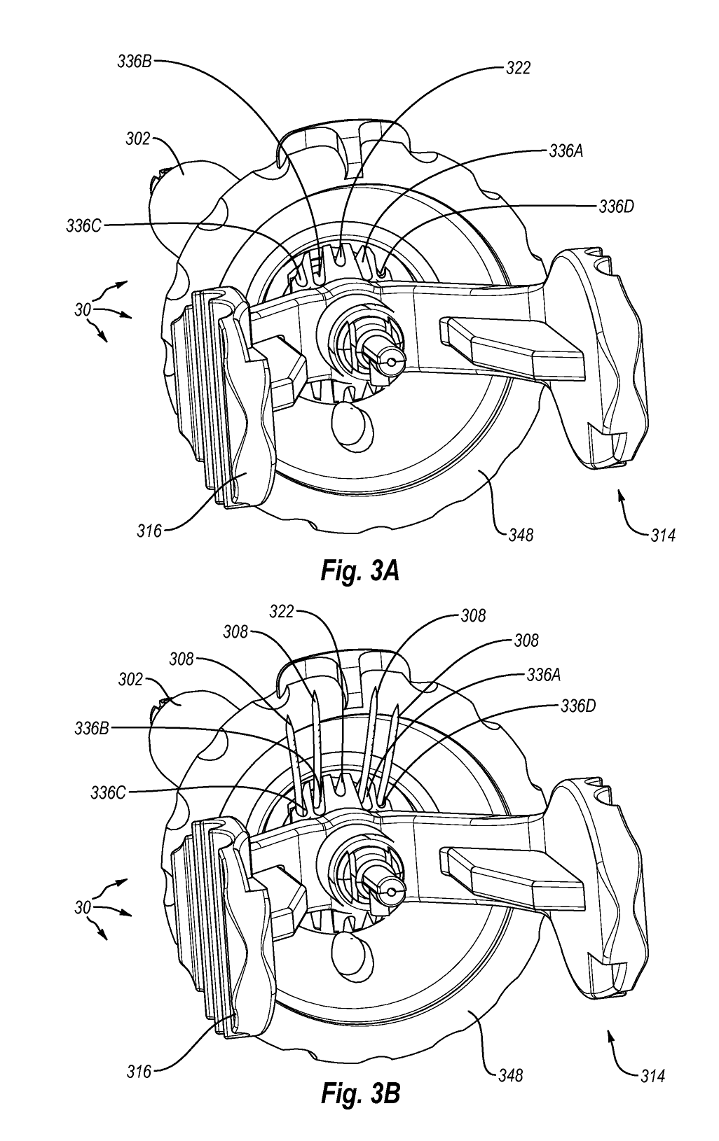

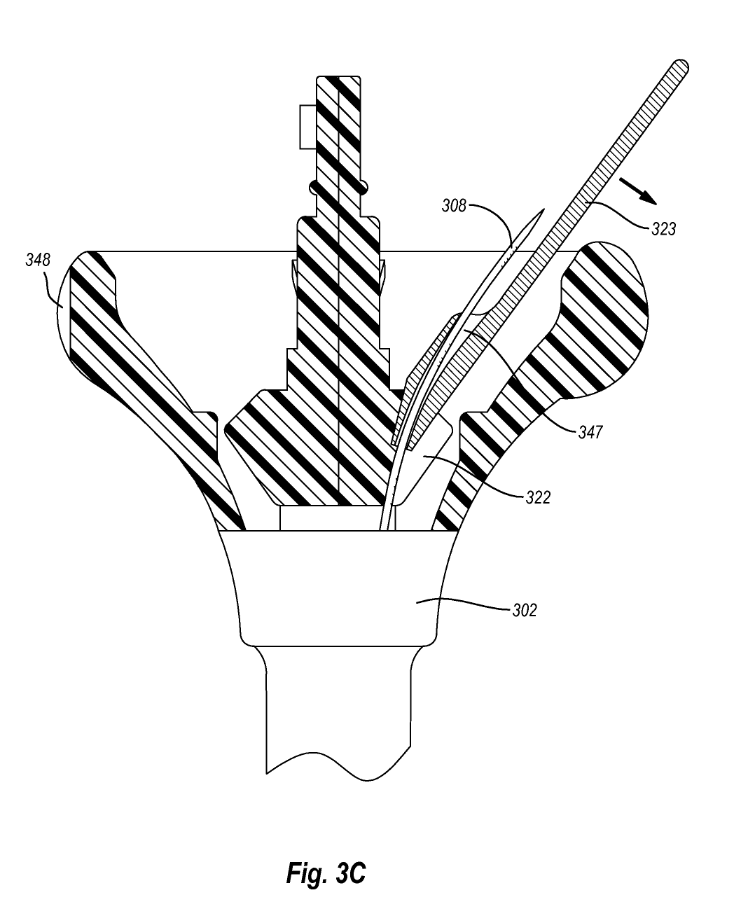

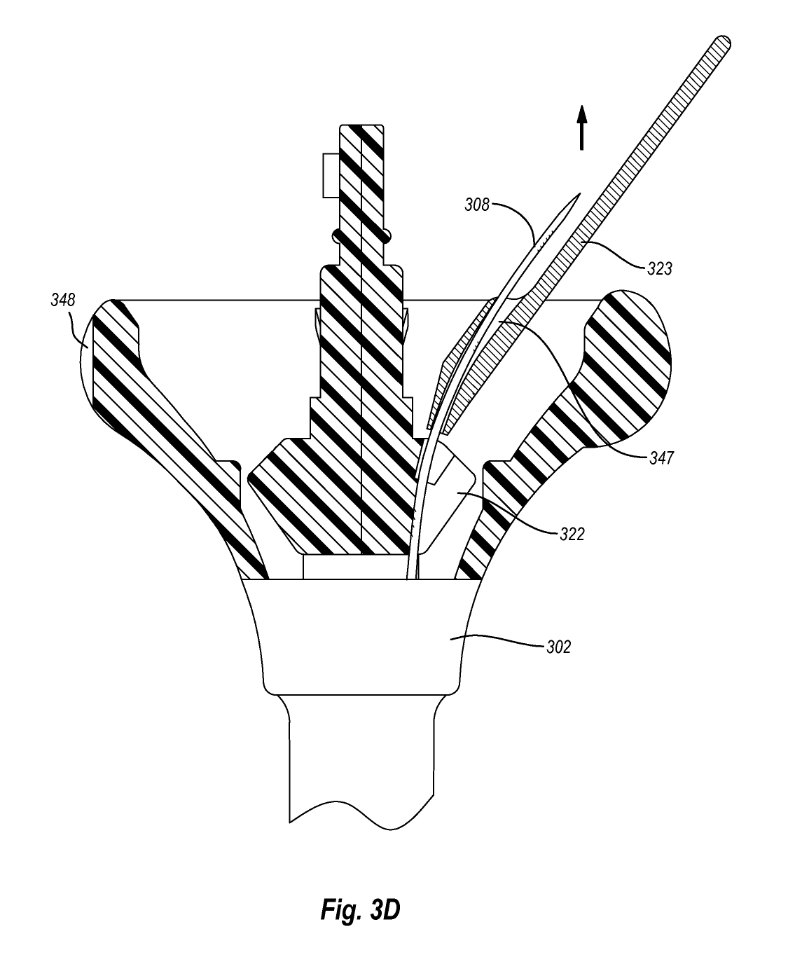

FIGS. 3A-3B illustrate a partial view of a suturing system 30. Suturing system 30 may be similar in many respects to suturing system 10 (including needle lumens). To the extent features or components of this configuration function in a manner similar to that as described above, such disclosure is hereby incorporated into the following additional configuration. Like structures and/or components are given like reference numerals. FIGS. 3A-3B primarily depict primarily proximal components of suturing system 30. The proximal components can be used to manipulate corresponding distal components (e.g., needles 308) as described with respect to suturing system 10.

Suturing system 30 may include guide body 302, a needle guide (not shown) secured to a distal end of guide body 302, and a flexible tube (not shown) secured to a distal end of the needle guide. A plurality of needles 308 (depicted in FIG. 3B) may be mounted with their distal ends in a support holster (not shown) and attached to a movable needle deployment shaft (also not shown). A handle assembly 314 may be attached to a proximal end of guide body 302. The handle assembly 314 may include a pair of interlock wings 316, proximal ends of needle lumens 336A, 336B, 336C, and 336D, and a handle (not shown). The handle may be attached to a proximal end of the needle deployment shaft and may be pulled proximally in order to draw needles 308 from the flexible tube, through a needle guide and into guide body 302 until the tips of needles 308 emerge from the proximal ends of needle lumens 336A, 336B, 336C, and 336D. As depicted, handle assembly 314 also includes anchor slot 322.

FIGS. 3C-3D illustrates a side cut away view of suturing system 30. Turning to FIG. 3C, anchor slot 322 is configured to receive needle removal tool 323. In general, needle removal tool 323 can be used to completely remove needles 308 from guide body 302. Needle removal tool 323 can include capture receiving slots 347 for each of needles 308. Needle removal tool 323 can be slid over needles 308 (with each of needles 308 going into a corresponding capture receiving slot 347) and secured into anchor slot 322. Capture receiving slots 347 are configured to grab and lock corresponding needles 308 when squeezed together (e.g., by a user's hand, a hemostat, etc.). Capture receiving slots 347 can include gripping features configured to help grasp the needles 308 such as adhesives, ridges, textured surfaces, magnets, or other suitable means.

Subsequently, force (somewhat radially and distally) can be applied near the proximal end of needle removal tool 323 to push needle removable tool 323 into contact with hub 348. After contacting hub 348, force can continue to be applied to needle removal tool 323. Leverage on hub 348 can be used to provide sufficient initial force for further proximal movement of needles 308. After the sufficient initial force, needle removal tool 323 can be squeezed (e.g. with a user's hand or hemostat) to grab and lock needles 308 in corresponding capture receiving slots 347. Turning to FIG. 3D, a user can then utilize further force to pull proximally to remove needle removal tool 323 and needles 308 from suturing system 30. Removal of needles 308 makes suture lengths available to the user.

Needle removal tool 323 may allow a user to exert an initial force (somewhat radially and distally) and/or further force (proximally) of about one quarter (0.25) pound-force to seventy (70) pound-force; about one (1) pound-force to sixty (60) pound-force; or about five (5) pound-force to forty (40) pound-force on needles 108 to overcome an initial resistance to proximal movement of needles 108 from guide body 102. In other embodiments, the needle removal tool 323 may allow a user to exert larger or smaller forces on needles 308. At least a portion of the sidewalls of capture receiving slots 347 may include gripping features such as ridges, textured surfaces, adhesives, magnets, or other features suitable to help grip needles 308. In other embodiments, the gripping features may be omitted.

Needle removal tool 323 may be made from polymers, polymeric composites, titanium, stainless steel, metal alloys, combinations thereof, or any other suitable materials

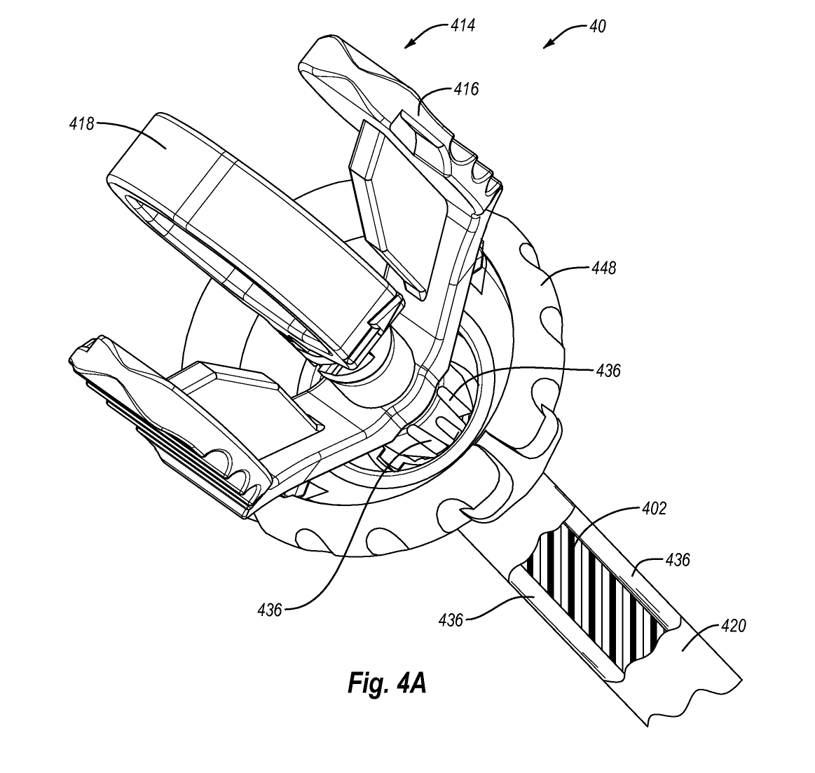

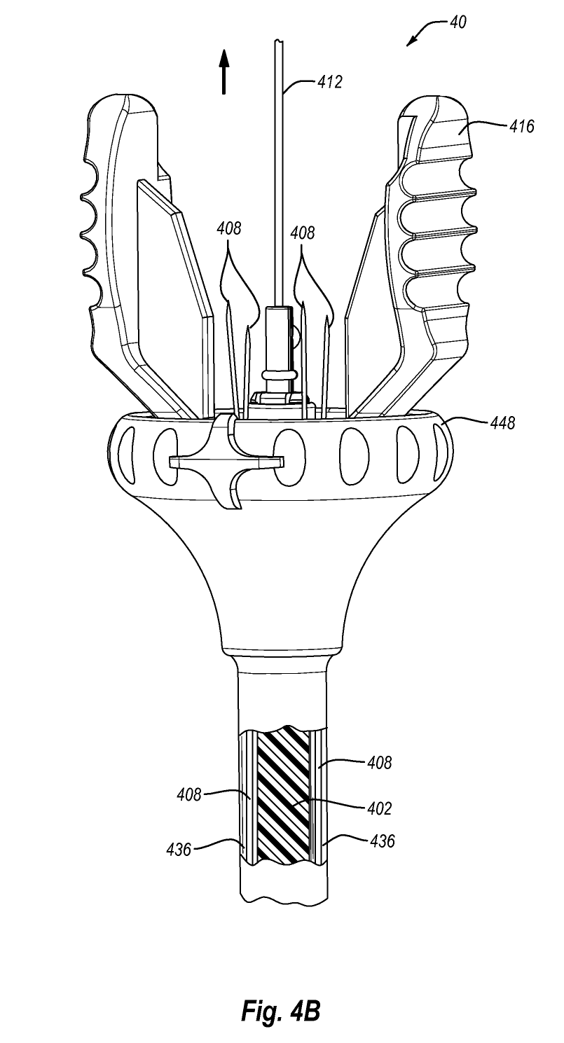

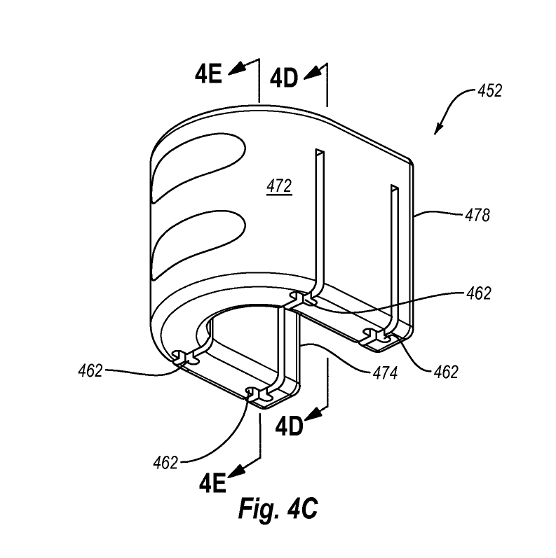

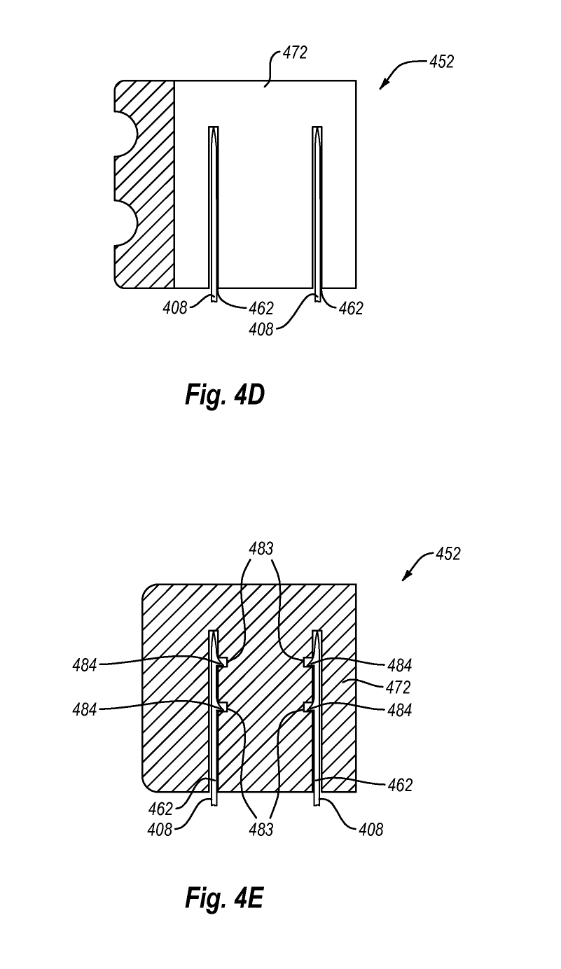

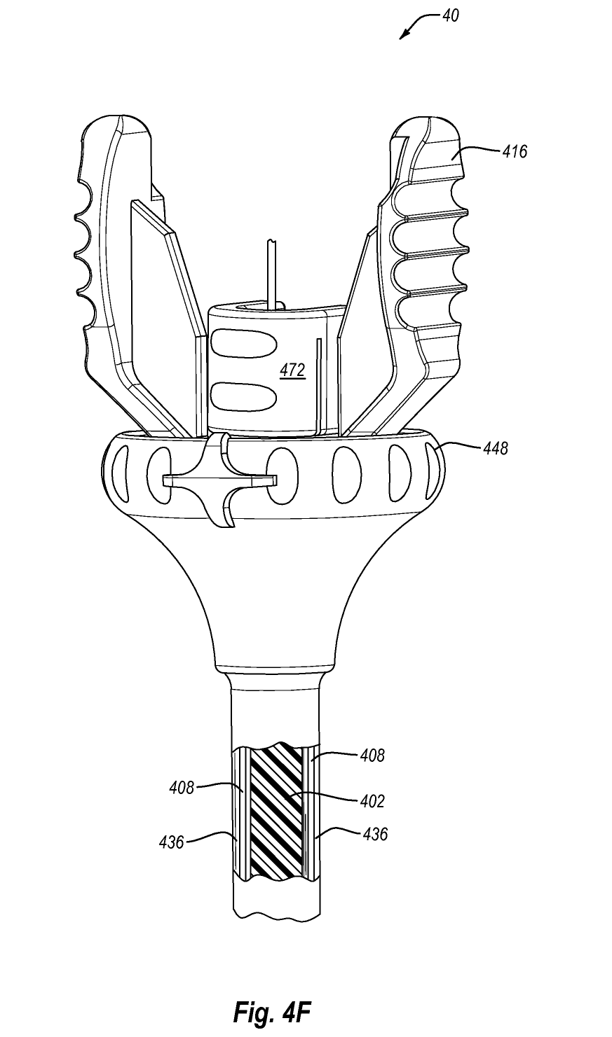

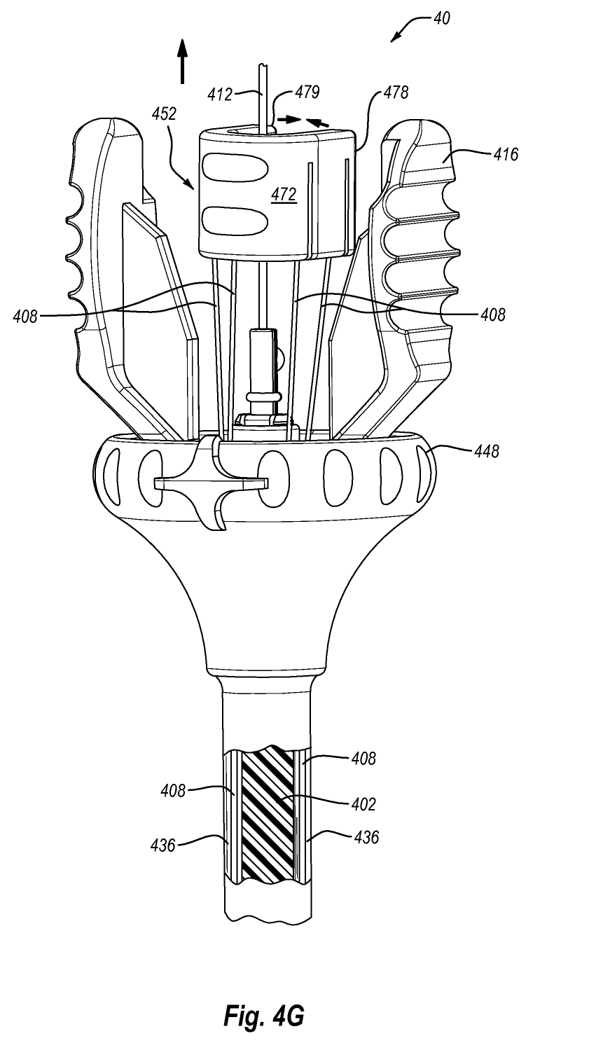

FIG. 4A is a partial side perspective view of the suturing system 40. The suturing system 30 may include guide body 402, a needle guide (not shown) secured to a distal end of the guide body 402, and a flexible tube (not shown) secured to a distal end of the needle guide. Sheath 420 may be rotatably received over guide body 402. A plurality of needles 408 (shown in FIG. 4B) may be mounted with their distal ends in a support holster (not shown) and attached to movable needle deployment shaft 312 (shown in FIG. 4B). Handle assembly 414 may be attached to a proximal end of guide body 402. Handle assembly 414 may include a pair of interlock wings 416 and handle 418. Handle 418 may be attached to a proximal end of needle deployment shaft 412 and may be pulled proximally in order to draw needles 408 from the flexible tube. Needles 408 can be pulled through the needle guide and into guide body 402 until the tips of needles 408 emerge from the proximal ends of needle lumens 436 in hub 448.

FIG. 4B illustrates a perspective view of suturing system 40. As depicted in FIG. 4B, the ends of needles 408 have been pulled some distance proximally from hub 448. A needle removal device can be used to remove needles 408 completely from suturing device 40.

FIG. 4C illustrates a perspective view of needle removal device 452 for removing needles from a suturing system, such as, for example, suturing system 40. As depicted, needle removal device 452 may include a generally U-shaped body 472. Needle removal device 452 may be made from polymers, polymeric composites, titanium, stainless steel, metal alloys, combinations thereof, or any other suitable materials.

U-shaped body 472 is wide enough to fit over needle deployment shaft 412. Thus, needle deployment shaft 412 may selectively be drawn proximally even after needle remove device has captured needles 408. Accordingly, needle deployment shaft 412 and needle removal device 452 may be configured to move axially relative to one another. Needle removal device 452 may be positioned at least partially within hub 448. In other embodiments, needle removal device 452 may be positioned proximal hub 448 or substantially within hub 448.

As depicted, body 472 includes a plurality of needle receptacles 462 formed in a bottom surface of body 472. Needle receptacles 462 may at least partially define lumens extending through the bottom surface of body 472 toward an upper surface of body 472. The lumen of needle receptacles 462 may have a circular, oval, triangular, or other suitable cross-sectional geometric shape. The lumens of one or more of needle receptacles 462 may have a constant diameter or a varying diameter. Needle receptacles 462 may be configured and positioned in body 472 to generally correspond to proximal ends of needle lumens 436 (in FIG. 4A).

This corresponding configuration may allow needles 408 to be selectively received within needle receptacles 462 when needle removal device 452 is positioned over needles 408. As depicted, four needle receptacles 462 may be formed in body 472. In other embodiments, three, five, six, or any other suitable numbers of needle receptacles 462 may be formed in body 472 in any suitable configuration. Generally, subsequently to capturing needs 408 in needle receptacles 462, ends 478 and 479 can be pressed towards one another causing needles 408 to be pinched within needle receptacles 462. Needle removal device 452 can then be moved proximally to remove needles 408 from guide body 402. Generally, ends 478 and 479 are spaced such that a user's hand or a hemostat can manipulate needle removal device with sufficient force to retain needles 408 within needle receptacles 462.

FIG. 4D illustrates a cross-sectional view of the needle removal device 452 taken along line 4D-4D. As depicted in FIG. 4D, the insides of needle receptacles 462 are smooth. When a sufficient length of needles 408 is captured in receptacles 462, ends 478 and 479 can be pressed towards one another causing needles 408 to be pinched and held within needle receptacles 462. In some embodiments, one or more needle receptacles 462 are filled with securing materials such as adhesives, epoxy, or other securing materials configured to help lock needles 408 in needle receptacles 462.

FIG. 4E illustrates a cross-sectional view of another needle removal device 452 taken along line 4E-4E. As depicted in FIG. 4E, body 472 may include a plurality of slots 483 inside body 472. Slots 483 may traverse one or more of needle receptacles 462 and form a locking edge configured to lock needles 408 in needle receptacles 462. Each needle receptacle 462 may include a pair of slots 483. In other embodiments, needle receptacles can have one, three, four, or any number of slots. Needles 408 can include notches 484 that catch in corresponding slots 483 to secure needles 408 within needle receptacles 462. One or more of slots 483 may be filled with securing materials such as adhesives, epoxy, or other securing materials configured to help lock needles 408 in needle receptacles 462.

Needles 408 can include notches 484 formed in the shaft portion of needs 408. Notches 484 may be formed circumferentially about the shaft portion, on one side of the shaft portion, or in any other part of the shaft portion of needles 408. Notches 484 may be configured to selectively lock into corresponding slots 483. For example, once needles 408 are received within needle receptacles 462, the edges of slots 483 may engage notches 484. Once engaged, needles 408 are locked in needle receptacles 462.

In various embodiments of needle removal device 452, needle receptacles 462 have a proximal end located within a solid portion of body 472. As such, the tips of needles 408 are housed within body 472 during needle removal. Keeping the tips of needles 408 within body 472 significantly reduces the chance of accidental sticks.

FIGS. 4F-4G illustrate a side view of the suturing system 40 with needles 408 captured (locked) in needle removal device 452. In the configuration depicted in FIG. 4F, needle removal device 452 can be drawn proximally to remove needles 408 from guide body 402 and make corresponding suture lengths available to a user. FIG. 4G depicts needle removal device 452 (and thus also needles 408) drawn proximally relative to the configuration in FIG. 4F.

To facilitate removal of needles 408 from guide body 402, handle 418 (depicted in FIG. 4A) may be drawn proximally relative to guide body 402 to proximally move needle deployment shaft 412. As depicted in FIGS. 4A and 4B, needle deployment shaft 412 may draw needles 408 proximally through needle lumens 436 of guide body 402 until needles 408 exit guide body 402 within hub 448.

As depicted in FIG. 4F, needle removal device 452 can be positioned to receive needles 408 in needle receptacles 462. When appropriate, pressure can be applied to push ends 478 and 479 towards one another. Depending on the configuration of needle removal device 452, the locking edges of slots 483 can engage notches 484 in the needles 408. Such a configuration may substantially lock needles 408 in needle receptacles 462. As depicted, the tips of needles 408 may be safely housed in the solid portion of body 472 such that risk of injury to a user or patient from the tips of needles 408 is reduced.

Turning to FIG. 4G, with needles 408 locked in needle receptacles 462, needle removal device 452 may be drawn proximally to pull needles 408 out of guide body 402. Once the needles 408 are removed from the guide body 402, suture lengths (not shown) attached to the needles 408 may be cut and the needles 408 may be disposed of. Such a configuration of the needle removal device 452 may allow a user to safely and quickly remove the needles 408 from the suturing system 40.

With needles captured in slots 462, needle removal device 452 may allow a user to exert a force in the proximal direction of about one quarter (0.25) pound-force to seventy (70) pound-force; about one (1) pound-force to sixty (60) pound-force; or about five (5) pound-force to forty (40) pound-force on needles 408. In other embodiments, needle removal device 452 may allow a user to exert larger or smaller forces on needles 408.

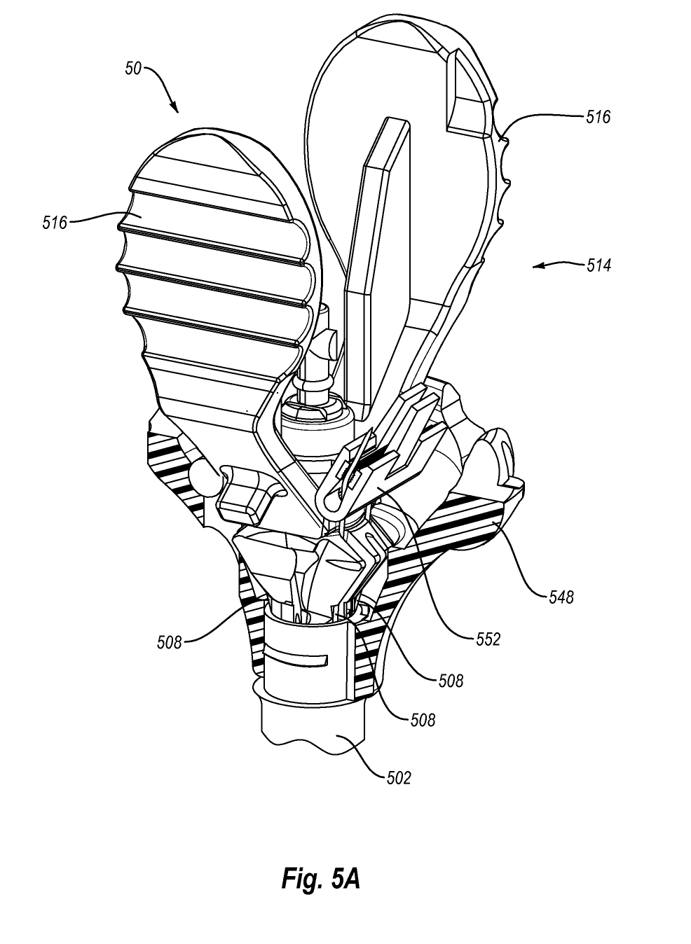

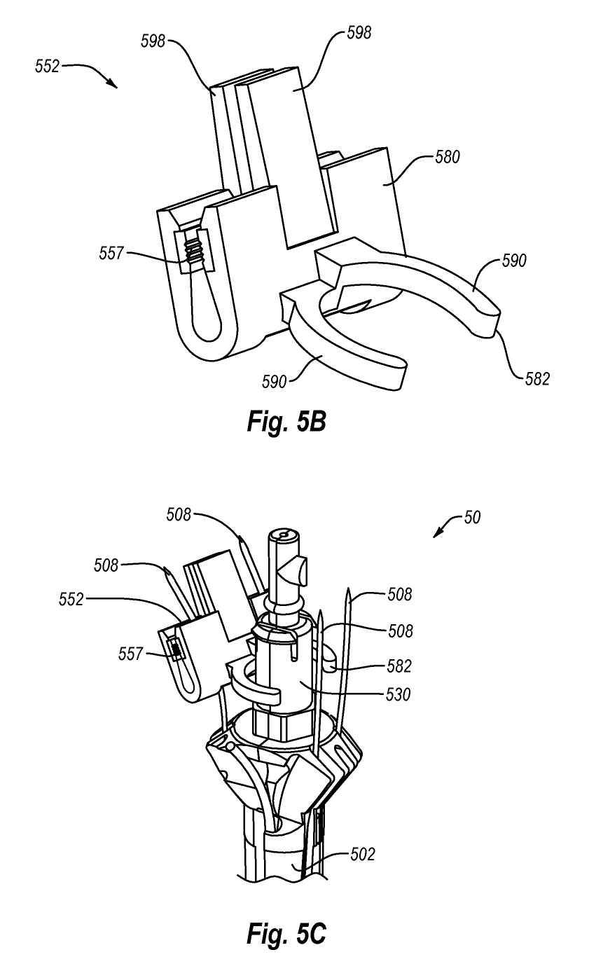

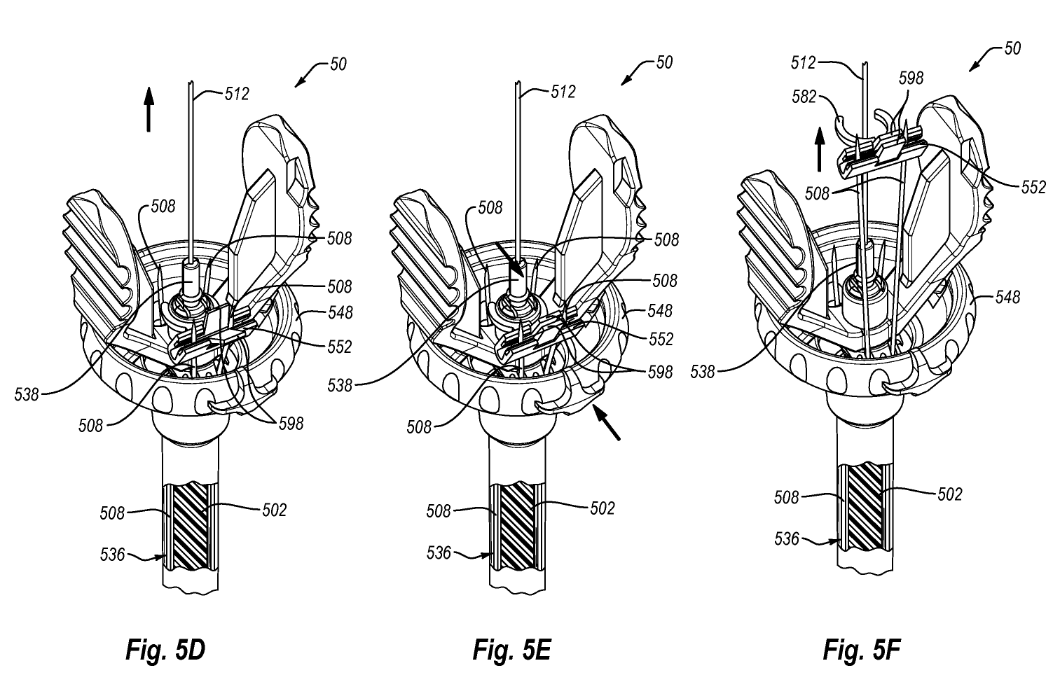

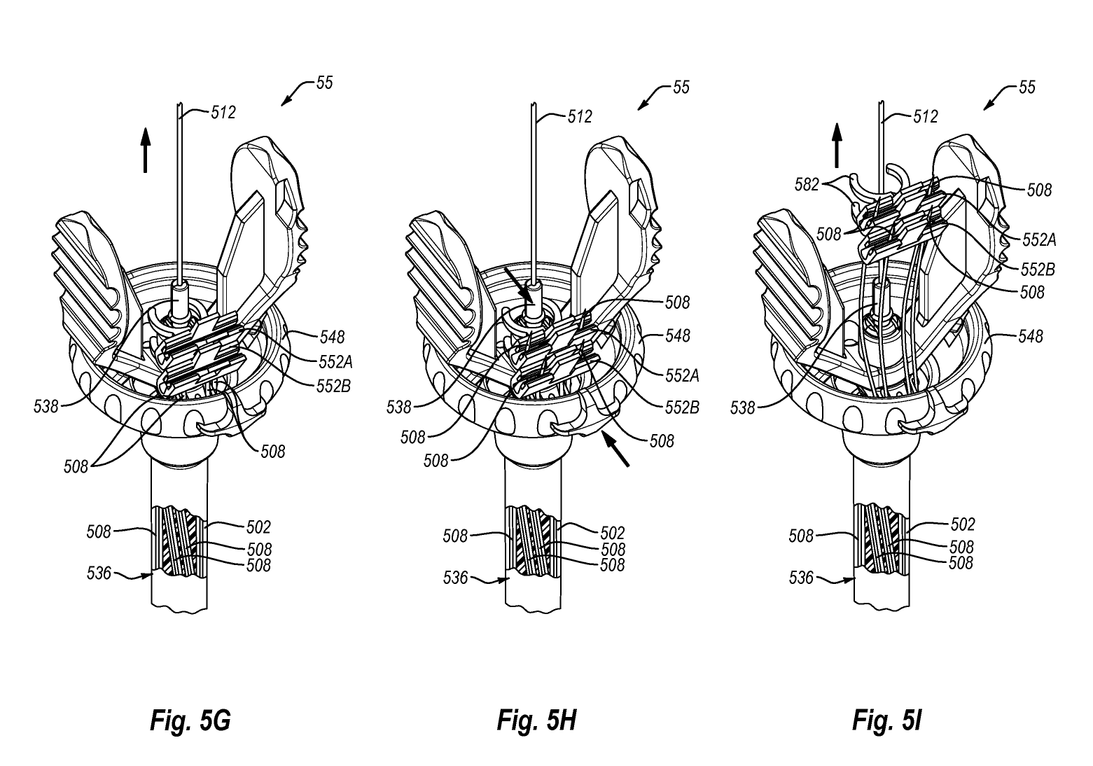

Turning to FIG. 5A, FIG. 5A depicts a side partial cross-section view a suturing system 50. Suturing system 50 may be similar in many respects to the suturing systems 10, 30, and 40 as previously described. To the extent features or components of this configuration function in a manner similar to that as described above, such disclosure is hereby incorporated into the following additional configuration. Like structures and/or components are given like reference numerals. Similar to suturing system 40, for ease of reference, the proximal portion of suturing system 50 is shown and described. The distal components may be manipulated by the proximal components as previously described.

Suturing system 50 may include guide body 502, a needle guide (not shown) secured to a distal end of guide body 502, and a flexible tube (not shown) secured to a distal end of the needle guide. A plurality of needles may be mounted with their distal ends in a support holster (not shown) and attached to a movable needle deployment shaft 512 (FIG. 5D). Handle assembly 514 may be attached to a proximal end of guide body 502. Handle assembly 514 may include a pair of interlock wings 516, needle removal device 552, and a handle. The handle may be attached to a proximal end of needle deployment shaft 512 and may be pulled proximally in order to draw needles 508 from the flexible tube, through the needle guide and into guide body 502 until needles 508 emerge from the guide body 502 within a hub 548.

Once needles 508 emerge within hub 548, needles 508 may be received within needle removal device 552. While one needle removal device 552 is shown, suturing system 50 may include two, three, or any suitable number of needle removal devices 552. For example, the suturing system 50 may include two needle removal devices 552 located on opposites sides of the guide body 502. Alternately, the suturing system 50 may include two needle removal devices 552 in tandem on the same side of guide body 502. Thus, a total of four needles may be received within the needle removal devices 552.

FIG. 5B is an isometric view of needle removal device 552. Turning to FIG. 5B, needle removal device 552 may include body member 580 and attachment ring 582. Needle removal device 552 may be formed from polymers, polymeric composites, titanium, stainless steel, combinations thereof, or any other suitable materials.

Body member 580 may include a pair of tabs 598. Tabs 598 may be connected to body member 580 by hinges. The hinges permit tabs 598 to be positioned toward or away from one another. Tabs 598 can be attached via adhesives, threadedly attaching, fastening with a fastener, welding, combinations of the foregoing, or another suitable technique. Tabs 598 may be configured to allow a user to move needle removal device 552 between the receiving and grasping positions. For example, a user may push tabs 598 apart to transition needle removal device 552 into the receiving position. On the other hand, a user may push or squeeze tabs 598 together to transition needle removal device 552 into the grasping position. The user may push or squeeze tabs 598 together with a user's fingers, a hemostat, or other suitable means. In the grasping position, teeth 557 can assist in securing needles within needle removal device 552.

In some embodiments, tabs 598 are configured to move the needle removal device between a receiving position and a grasping position. In the receiving position, any needles are moveable within one or more needle receptacles. In the grasping position, tabs 598 flex body member 580 such that one or more grooves (e.g., of teeth 557) grasp any needles between opposing sidewalls of the one or more grooves to secure the needles within the plurality of needle receptacles.

Thus, needle removal device 552 may include locking features configured to selectively lock needle removal device 552 in the grasping position. For example, one of tabs 598 may include one or more locking arms configured to rotate over other tab 598 when needle removal device 552 is in the grasping position to hold tabs 598 together. Other tab 598 may include one or more grooves (e.g., teeth 557) configured to receive and/or secure the one or more locking arms on other tab 598. In other embodiments, locking features may include a hook member configured to hold tabs 598 together in the grasping position. In other embodiments, the locking features may be omitted.

FIG. 5C depicts needle removal device 552 attached to suturing system 50. As depicted, attachment arms 590 are configured to selectively attach needle removal device 552 to stem 538 proximal to guide body 502. Attachment arms 590 may form at least a portion of circle having a diameter configured to generally correspond to an outer diameter of stem 538. Generally, attachment arms 590 may be configured to flex apart such that attachment ring 582 may be removably attached to stem 538 by moving attachment ring 582 in a direction substantially traverse to the stem 538. In other embodiments, attachment arms 590 may be resiliently biased, substantially rigid, or they may have any other configuration suitable to attach and remove attachment ring 582 from stem 538.

FIGS. 5D-5F illustrates a perspective view of suturing system 50 shown with needles captured in needle removal device 552. Generally, needle removal device 552 may be moveable between a receiving position, wherein tabs 598 are moved apart such that needles 508 may extend therebetween, and a grasping position, wherein tabs 598 are moved together to grasp or pinch needles in teeth 557.

As depicted in FIG. 5D, needle removal device 552 can be moved distally over needles 508. Needle removal device 552 may have needle receptacles configured and positioned to generally correspond to needle lumens 536 at the proximal end of guide body 502. Such a configuration may allow needles 508 to be received within the needle receptacles when needles 508 exit needle lumens 536. The one or more needle receptacles may include grasping portions formed on the inside of body member 580. In other embodiments, the grasping portions may include one or more gripping features such as ridges (e.g., teeth 557), textured surfaces, contoured surfaces, adhesive, magnets, or other features suitable to enhance the grip of needle removal device 552 on needles 508.

In addition, body member 580 may be substantially rigid to improve compliance of needle removal device 552 and to enhance the grip of needle removal device 552 on needles 508. Such a configuration may allow a user to exert a force in the proximal direction on needles 508 to overcome an initial resistance to removal of needles 508 from guide body 502. For example, in the grasping position, needle removal device 552 may allow a user to exert a force in the proximal direction of about one quarter (0.25) pound-force to seventy (70) pound-force; about one (1) pound-force to sixty (60) pound-force; or about five (5) pound-force to forty (40) pound-force on needles 508. In other embodiments, needle removal device 552 may allow a user to exert larger or smaller forces on needles 508.

A method for removing needles 508 from suturing system 50 is described with respect to FIGS. 5D-5F. While the method is illustrated using suturing system 50 and needle removal device 552, it will be appreciated that the described method may utilize any other needle removal device and/or suturing system disclosed herein. Certain exemplary steps of removing needles 508 from suturing system 50 are shown and described. However, it is to be appreciated that the method may follow delivery of needles and suture lengths through body tissue or a vessel wall by the suturing system 50 including any of the steps previously described and/or illustrated.

Referring now to FIG. 5D, to deploy needles 508, handle 518 (e.g., similar to handle 118 in FIG. 1A) may be drawn proximally relative to guide body 502 to proximally move needle deployment shaft 512. As depicted, needle deployment shaft 512 may draw needles 508 proximally through the needle lumens 536 of guide body 502 until needles 508 exit guide body 502 within hub 548. As needles 508 exit the guide body 502, needles 508 may be received within needle removal device 552 while needle removal device 552 is in the receiving position as shown.

Turning to FIG. 5E, tabs 598 may be squeezed or pushed together to move needle removal element 552 into the grasping position. Tabs 598 may be squeezed or pushed together with a user's fingers, a hemostat, or any other suitable means. In the grasping position, needles 508 may be grasped or pinched by needle removal device 552, such as, for example, within gripping features configured to help grasp needles 508 such as adhesives, ridges, textured surfaces, magnets, or other suitable means.

Turning to FIG. 5F, needle removal device 552 may be moved proximally relative to guide body 502. With needle removal device 552 in the grasping position, needle removal device 552 may allow a user to exert a force in the proximal direction of about one quarter (0.25) pound-force to seventy (70) pound-force; about one (1) pound-force to sixty (60) pound-force; or about five (5) pound-force to forty (40) pound-force on the needles 508. In other embodiments, needle removal device 552 may allow a user to exert larger or smaller forces on needles 508.

Attachment ring 582 may be removed from stem 538 by moving the attachment ring 582 axially or substantially traverse relative to stem 538. Proximal movement of needle removal device 552, in turn, may continue to remove needles 508 from guide body 502. Once needles 508 are removed from guide body 502, suture lengths (not shown) attached to needles 508 may be cut and needles 508 may be disposed of.

FIGS. 5G-5I illustrates a perspective view of suturing system 55 with needles 508 captured in needle removal devices 552. Generally, needle lumens 536 are configured similar to needle lumens 136 (depicted in FIGS. 1B-2C). As such, needles 508 exit hub 548 in closer proximity to one another. For example, as depicted in FIG. 5G, needles 508 come out essentially on the same side of guide body 502.

Referring now to FIG. 5G, to deploy needles 508, handle 518 (e.g., similar to handle 118 in FIG. 1A) may be drawn proximally relative to guide body 502 to proximally move needle deployment shaft 512. As depicted, needle deployment shaft 512 may draw needles 508 proximally through needle lumens 536 of guide body 502 until needles 508 exit guide body 502 within hub 548. As needles 508 exit guide body 502, needles 508 may be received within needle removal devices 552A and 552B while needle removal devices 552A and 552B are in the receiving position as shown.

Turning to FIG. 5H, tabs 598 may be squeezed or pushed together to move the needle removal devices 552A and 552B into the grasping position. Tabs 598 may be squeezed or pushed together with a user's fingers, a hemostat, or any other suitable means. In the grasping position, needles 508 may be grasped or pinched by needle removal device 552, such as, for example, within gripping features configured to help grasp needles 508 such as adhesives, ridges, textured surfaces, magnets, or other suitable means.

Turning to FIG. 5I, needle removal devices 552A and 552B may be moved proximally relative to guide body 502. With needle removal devices 552A and 552B in the grasping position, needle removal devices 552A and 552B may allow a user to exert a force in the proximal direction of about one quarter (0.25) pound-force to seventy (70) pound-force; about one (1) pound-force to sixty (60) pound-force; or about five (5) pound-force to forty (40) pound-force on the needles 508. In other embodiments, needle removal devices 552A and 552B may allow a user to exert larger or smaller forces on needles 508.

Attachment rings 582 may be removed from stem 538 by moving attachment ring 582 axially or substantially traverse relative to stem 538. Proximal movement of needle removal devices 552A and 552B, in turn, may continue to remove needles 508 from guide body 502. Once needles 508 are removed from guide body 502, suture lengths (not shown) attached to needles 508 may be cut and needles 508 may be disposed of.

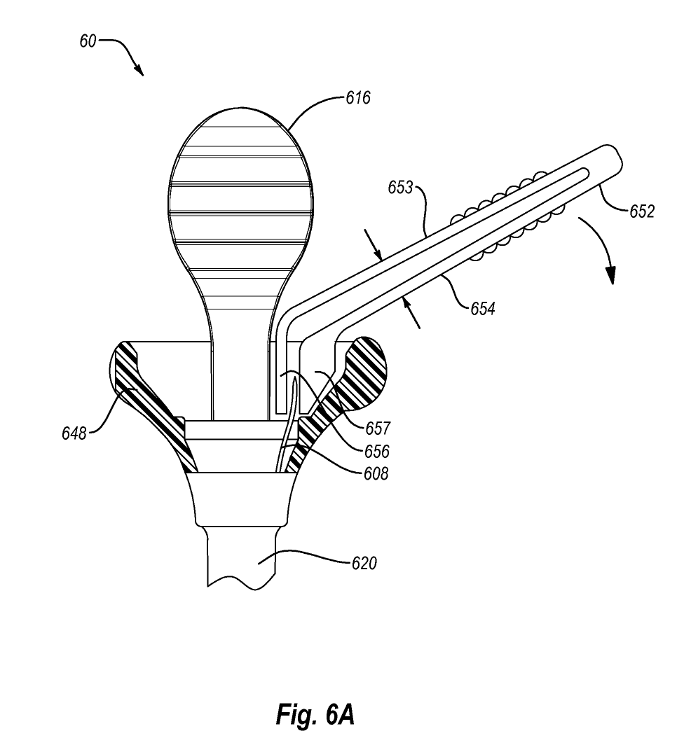

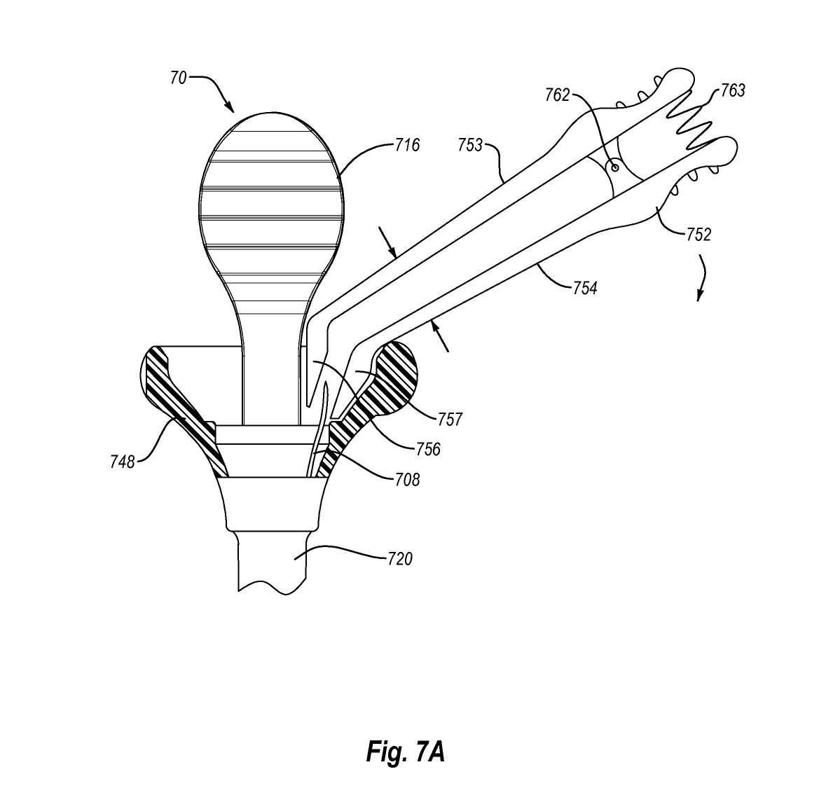

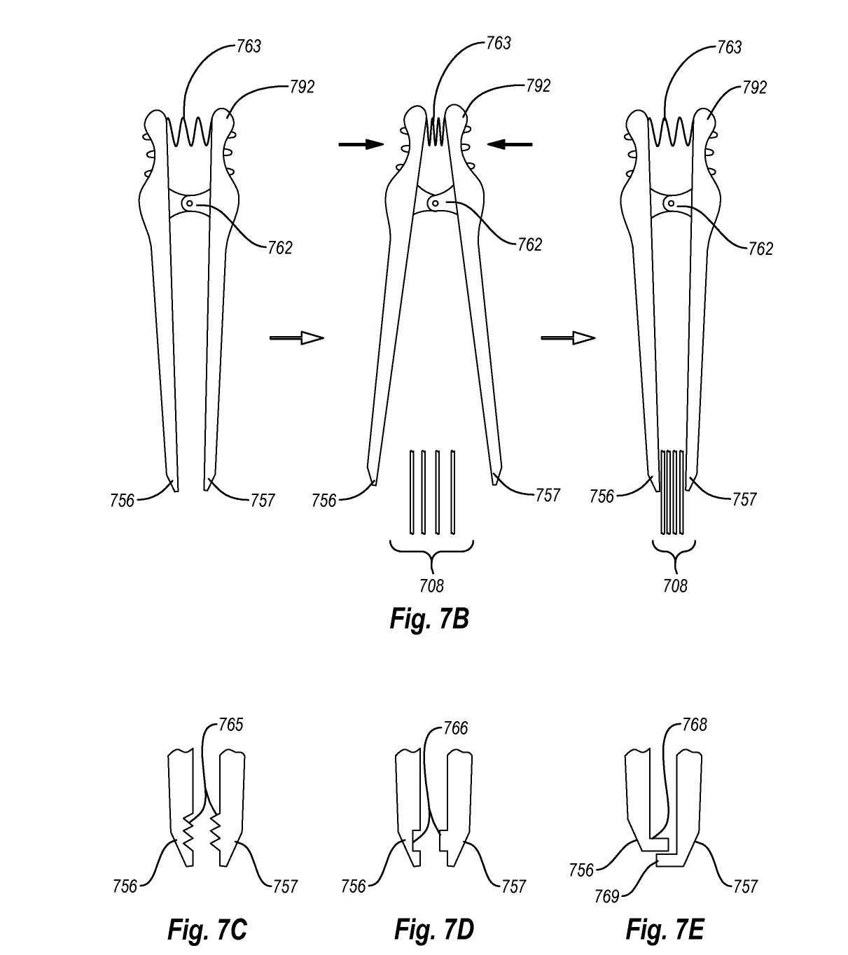

FIG. 6A depicts needle removal tool 652. Needle removal tool 652 can be used to completely remove needles 608 from guide body 602. Needle removal tool 652 can be slid over a needle 608. Members 653 and 654 can be squeezed or pushed together with a user's fingers, a hemostat, or any other suitable means. When members 653 and 654 are squeezed or pushed together needle 608 can be captured in ends 656 and 657. Ends 656 and 657 can include gripping features configured to help grasp the needles 608 such as adhesives, ridges, textured surfaces, magnets, or other suitable means (including those in FIGS. 7C-7E).

Subsequently, force (somewhat radially and distally) can be applied near the proximal end of needle removal tool 652 to push needle removable tool 652 into contact with hub 648. After contacting hub 648, force can continue to be applied to needle removal tool 652. Leverage on hub 648 can be used to provide sufficient initial force for further proximal movement of needle 608. After the sufficient initial force, a user can then pull proximally to remove needle removal tool 652 and needle 608 from suturing system 60. Removal of needle 608 makes suture lengths available to the user.

Needle removal tool 652 can be configured to remove one or more needles 608 at a time. For example, in configurations similar to FIGS. 1B-2C, needle removal tool 652 can be configured to grab and remove four needles on one side of a guide body.

FIG. 7A depicts needle removal tool 752. As depicted, needle removal tool 752 includes hinge 762 and spring 763. Needle removal tool 752 can be used to completely remove needles 708 from guide body 702. Pressure can be applied to members 753 and 754 proximal of hinge 762 (e.g., with a user's fingers, a hemostat, or any other suitable means) to overcome the force of spring 763. The applied force can cause ends 756 and 757 to separate. In this separated configuration, needle removal tool 752 can be slid over a needle 708.

Subsequently, pressure applied to members 753 and 754 proximal of hinge 762 can be removed. In response, the force of spring 763 causes ends 756 and 757 to close on needle 708. In some embodiments, the force of spring 763 is sufficient to hold needle 708 without slipping. In other embodiments, members 653 and 654 can be squeezed or pushed together distal of hinge 762 (e.g., with a user's fingers, a hemostat, or any other suitable means) to assist in gripping needle 708. In either embodiment, members 753 and 754 are squeezed or pushed together captured needle 708 in ends 756 and 757.

Subsequently, force (somewhat radially and distally) can be applied near the proximal end of needle removal tool 752 to push needle removable tool 752 into contact with hub 748. After contacting hub 748, force can continue to be applied to needle removal tool 752. Leverage on hub 748 can be used to provide sufficient initial force for further proximal movement of needle 708. After the sufficient initial force, a user can then pull proximally to remove needle removal tool 752 and needle 708 from suturing system 70. Removal of needle 708 makes suture lengths available to the user.

Needle removal tool 752 can be configured to remove one or more needles 708 at a time. For example, in configurations similar to FIGS. 1B-2C, needle removal tool 752 can be configured to grab and remove four needles on one side of a guide body.

FIG. 7B illustrates another needle removal tool 792. Needle removal device 792 is similar to needle removal device 752 but is of a straighter configuration. In FIG. 7B, spring 763 can be of sufficient strength to hold needles 708 during removal (i.e., pulling needle removal tool 792 proximally).

For either of needle removal tools 752 and 792, ends 756 and 757 can include gripping features configured to help grasp the needles 708 such as adhesives, ridges, textured surfaces, magnets, or other suitable means (including those in FIGS. 7C-7E). FIG. 7C depicts ends 756 and 757 having a number of teeth 765. FIG. 7D depicts ends 756 and 757 having a slot and tooth arrangement 766. FIG. 7E depicts ends 756 and 757 or different lengths 768 and 769 respectively. In any of these configurations, ends 756 and 757 assist in gripping and holding a needle or needles in place during removal of the needle or needles.

Accordingly, needle removal tools 652 and 752 may allow a user to exert an initial force (somewhat radially and distally) and/or further force (proximally) of about one quarter (0.25) pound-force to seventy (70) pound-force; about one (1) pound-force to sixty (60) pound-force; or about five (5) pound-force to forty (40) pound-force on the needles 108 to overcome an initial resistance to proximal movement of needles from a guide body 102. In other embodiments, needle removal tools 652 and 752 may allow a user to exert larger or smaller forces on needles.

In some embodiments of the invention, needle removal devices may be configured to secure needles for removal from suturing systems by at least partially deforming the needles as described in U.S. patent application, entitled “Needle Harvesting Devices, Systems and Methods,” attorney docket number 16497.245, filed on the same day, the disclosure of which is incorporated herein in its entirety.

Any embodiments of suturing devices, needle removal devices, and the like may include a material made from any of a variety of known suitable biocompatible materials, such as a biocompatible shape memory material (SMM). For example, the SMM may be shaped in a manner that allows for the needle removal device to automatically move from the receiving position to the grasping position when needles are received within the needle receptacles. SMMs have a shape memory effect in which they may be made to remember a particular shape. Once a shape has been remembered, the SMM may be bent out of shape or deformed and then returned to its original shape by unloading from strain or heating. Typically, SMMs may be shape memory alloys (SMA) comprised of metal alloys, or shape memory plastics (SMP) comprised of polymers. The materials may also be referred to as being superelastic.

Usually, an SMA may have an initial shape that may then be configured into a memory shape by heating the SMA and conforming the SMA into the desired memory shape. After the SMA is cooled, the desired memory shape may be retained. This allows for the SMA to be bent, straightened, twisted, compacted, and placed into various contortions by the application of requisite forces; however, after the forces are released, the SMA may be capable of returning to the memory shape. The main types of SMAs are as follows: copper-zinc-aluminum; copper-aluminum-nickel; nickel-titanium (NiTi) alloys known as nitinol; nickel-titanium platinum; nickel-titanium palladium; and cobalt-chromium-nickel alloys or cobalt-chromium-nickel-molybdenum alloys known as elgiloy alloys. The temperatures at which the SMA changes its crystallographic structure are characteristic of the alloy, and may be tuned by varying the elemental ratios or by the conditions of manufacture.

For example, the primary material of needle removal device may be of a NiTi alloy that forms superelastic nitinol. Also, additional materials may be added to the nitinol depending on the desired characteristic. The alloy may be utilized having linear elastic properties or non-linear elastic properties.

An SMP is a shape-shifting plastic that may be fashioned into the needle receptacles of the base member in accordance with the present disclosure. Also, it may be beneficial to include at least one layer of an SMA and at least one layer of an SMP to form a multilayered body; however, any appropriate combination of materials may be used to form a multilayered device. When an SMP encounters a temperature above the lowest melting point of the individual polymers, the blend makes a transition to a rubbery state. The elastic modulus may change more than two orders of magnitude across the transition temperature (Ttr). As such, an SMP may be formed into a desired shape of an endoprosthesis by heating it above the Ttr, fixing the SMP into the new shape, and cooling the material below Ttr. The SMP may then be arranged into a temporary shape by force and then resume the memory shape once the force has been released. Examples of SMPs include, but are not limited to, biodegradable polymers, such as oligo(ε-caprolactone)diol, oligo(ρ-dioxanone)diol, and non-biodegradable polymers such as, polynorborene, polyisoprene, styrene butadiene, polyurethane-based materials, vinyl acetate-polyester-based compounds, and others yet to be determined. As such, any SMP may be used in accordance with the present disclosure.

The needle receptacles and the like may have at least one layer made of an SMM or suitable superelastic material and other suitable layers that can allow the needle receptacles to automatically grasp onto the needles.

Also, the needle removal devices, the needle receptacles or other aspects or components of the system may be comprised of a variety of known suitable deformable materials, including stainless steel, silver, platinum, tantalum, palladium, nickel, titanium, nitinol, nitinol having tertiary materials (U.S. 2005/0038500, which is incorporated herein by reference, in its entirety), niobium-tantalum alloy optionally doped with a tertiary material (U.S. 2004/0158309, 2007/0276488, and 2008/0312740, which are each incorporated herein by reference, in their entireties) cobalt-chromium alloys, or other known biocompatible materials. Such biocompatible materials may include a suitable biocompatible polymer in addition to or in place of a suitable metal. The polymeric needle removal device may include biodegradable or bioabsorbable materials.

In one embodiment, the needle removal device and/or needle receptacles may be made from a superelastic alloy such as nickel-titanium or nitinol, and includes a ternary element selected from the group of chemical elements consisting of iridium, platinum, gold, rhenium, tungsten, palladium, rhodium, tantalum, silver, ruthenium, or hafnium. The added ternary element improves the radiopacity of the nitinol knot replacement element. The nitinol needle removal device has improved radiopacity yet retains its superelastic and shape memory behavior and further maintains a thin body thickness for high flexibility.

In one embodiment, the needle removal device and/or the needle receptacles may be made at least in part of a high strength, low modulus metal alloy comprising Niobium, Tantalum, and at least one element selected from the group consisting of Zirconium, Tungsten, and Molybdenum.

In further embodiments, the needle removal device and/or the needle receptacles may be made from or be coated with a biocompatible polymer. Examples of such biocompatible polymeric materials may include hydrophilic polymer, hydrophobic polymer, biodegradable polymers, bioabsorbable polymers, and monomers thereof. Examples of such polymers may include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, polyethylenes, polypropylenes, polyaliphatics, polyvinylalcohols, polyvinylacetates, hydrophobic/hydrophilic copolymers, alkylvinylalcohol copolymers, ethylenevinylalcohol copolymers (EVAL), propylenevinylalcohol copolymers, polyvinylpyrrolidone (PVP), combinations thereof, polymers having monomers thereof, or the like.

The coatings can also be provided on the system or components thereof to facilitate the loading or delivery of beneficial agents or drugs, such as therapeutic agents, pharmaceuticals and radiation therapies.

The invention is susceptible to various modifications and alternative means, and specific examples thereof have been shown by way of example in the drawings and are herein described in detail. It should be understood, however, that the invention is not to be limited to the particular systems or methods disclosed, but to the contrary; the invention is to cover all modifications, equivalents, and alternatives falling within the spirit and scope of the claims.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A suturing device, the suturing device comprising:
a guide body having a proximal end and a distal end;
a shaft having a proximal end and a distal end, the shaft slidably mounted in the guide body;
a plurality of needles removably carried near the distal end of the shaft, each of the plurality of needles including a shank and a sharpened tip, the shank carried on the shaft and the sharpened tip disposed toward the proximal end of the shaft;
one or more lengths of suture secured to and extending between the plurality of needles; and
a plurality of needle lumens, each needle lumen having a distal end opening on the guide body and a corresponding proximal end opening on the guide body, each needle lumen configured to receive a corresponding one of the plurality of needles at a distal end opening and transfer the needle to the corresponding proximal end opening as the plurality of needles are drawn proximally by the shaft, each of the distal end openings located radially outwardly from the center of the guide body, each of the proximal end openings located radially outwardly from the center of the guide body, the proximal end openings clustered together in closer proximity to one another relative to the distal openings, at least one needle lumen configured with a needle path to move a corresponding needle partially circumferentially around the guide body when the plurality of needles are drawn proximally by the shaft.

2. The suturing device of claim 1, further comprising a needle receiving element attached to the guide body, the needle receiving element for receiving each of the plurality of needles and passing each of the plurality of needles into the corresponding needle lumen.

3. The suturing device of claim 1, further comprising one or more suture lumens configured to receive at least a portion of the one or more lengths of sutures.

4. The suturing device of claim 1, further comprising a first needle removal device, the first needle removal device comprising:
one or more needle receptacles, the one or more needle receptacles positioned and configured to generally correspond to the proximal ends of a first one or more of the plurality of needle lumens of the suturing device, the needle receptacles being further configured to selectively receive and grasp onto a first one or more needles extending proximally from the first one or more needle lumens of the suturing device.

5. The suturing device of claim 4, wherein the one or more needle receptacles comprises a plurality of needle receptacles, each of the plurality of needle receptacles corresponding to one of the plurality of needle lumens.

6. The suturing device of claim 4, wherein the plurality of needles comprises four needles; and
wherein the plurality of needle lumens comprises four needle lumens, each of the four needle lumens corresponding to one of the plurality of needles, two of the four needle lumens configured with a needle path to move a corresponding first two of the four needles partially circumferentially around the guide body so that the first two needles are in closer proximity to other two of the four needles at the proximal end of the four needle lumens.

7. The suturing device of claim 4, further comprising a second needle removal device, the second needle removal device comprising:
one or more needle receptacles, the one or more needle receptacles positioned and configured to generally correspond to the proximal ends of a second one or more of the plurality of needle lumens of the suturing device, the needle receptacles being further configured to selectively receive and grasp onto a second one or more needles extending proximally from the second one or more needle lumens of the suturing device, the second one or more needles differing from the first one or more needles.

8. A needle removal device comprising:
a body member configured to be positioned on one or more proximally exposed needles of a suturing system, the one or more needles exposed from corresponding needle lumens on a proximal portion of the suturing system; and
one or more needle receptacles at least partially defined by the body member, the one or more needle receptacles being positioned and configured to generally correspond to the needle lumens of the suturing device, the needle receptacles being further configured to selectively receive and grasp onto the proximally exposed one or more needles.

9. The needle removal device of claim 8, wherein the body member is further configured to allow a user to selectively exert a force in a proximal direction of about one quarter (0.25) pound-force to seventy (70) pound-force on the proximally exposed one or more needles to remove the proximally exposed one or more needles from the suturing system.

10. The needle removal device of claim 9, wherein the needle receptacles include one or more gripping features configured to enhance the grasp of the needle receptacles on the one or more needles.

11. The needle removal device of claim 8, wherein the body member is at least semi-flexible and the one or more needle receptacles are formed in a bottom surface of the body member, and further comprising:
one or more grooves formed in an upper surface of the body member, the one or more grooves being in communication with the one or more needle receptacles; and
a pair of tabs attached to opposite ends of the body member, the tabs being configured to move the needle removal device between a receiving position, wherein at least a portion of the one or more needles are moveable within the one or more needle receptacles, and a grasping position, wherein the tabs flex the body member such that the one or more grooves grasp the one or more needles between opposing sidewalls of the one or more grooves to secure the one or more needles within the one or more needle receptacles.

12. The needle removal device of claim 11, wherein the grasping position is further configured to allow a user to exert a force in a proximal direction of about one quarter (0.25) pound-force to thirty (30) pound-force on the one or more needles to remove the one or more needles from the suturing system.

13. The needle removal device of claim 8, wherein the body member comprises a pair of attachment arms forming an attachment ring, the attachment ring configured to removably attach to the suturing system.

14. The needle removal device of claim 13, further comprising one or more tabs attached to the body member, the one or more tabs being configured to move the body member between a receiving position and a grasping position.

15. The needle removal device of claim 8, wherein the needle removal device is configured to fit into an anchor slot of the suturing system prior to needle removal.

16. The needle removal device of claim 8, wherein the one or more needle receptacles each includes one or more lateral slots, the one or more lateral slots configured to catch corresponding notches on the one or more needles to secure the one or more needles in the one or more needle receptacles.

17. A method for removing one or more needles from a suturing system, the suturing device having a hub, the method comprising:
drawing a plurality of needles proximally through the suturing system until at least tips of the plurality of needles exit from the proximal end of the suturing system;
positioning a needle removal device substantially adjacent to a proximal end of the suturing system;
receiving at least the tips of one or more of the needles within needle receptacles formed within the needle removal device;
securing at least the tips of the one or more needles within the needle receptacles; and
moving the needle removal device proximally relative to the suturing system to remove the one or more needles from the suturing system.

18. The method of claim 17, further comprising:
prior to moving the needle removal device proximally, moving the needle removal device radially and distally into contact with the hub;
applying further force radially and distally to the proximal end of the needle removal device to use the hub as leverage for overcoming an initial amount of force for moving the one or more needles proximally.

19. The method of claim 17, further comprising:
positioning a second needle removal device substantially adjacent the proximal end of the suturing system;
receiving at least the tips of a second one or more of the needles within needle receptacles formed within the second needle removal device;
securing at least the tips of the second one or more needles within the second needle receptacles; and
moving the needle removal device proximally relative to the suturing system to remove the second one or more needles from the suturing system.

20. The method as recited in claim 17, wherein
receiving at least the tips of one or more of the needles within needle receptacles formed within the needle removal device comprises receiving the tips of the plurality of needles within needle receptacles formed within the needle removal device
securing at least the tips of the one or more needles within the needle receptacles comprises securing at least the tips of the plurality of needles within the needle receptacles; and
moving the needle removal device proximally relative to the suturing system to remove the one or more needles from the suturing system comprises moving the needle removal device proximally relative to the suturing system to remove the plurality of needles from the suturing system.
